(19) United States
(12) Patent Application Publication
(10) Pub. No.: US 2018/0207087 A1
(43) Pub. Date: Jul. 26, 2018

(54) KIT AND AESTHETIC SYSTEM FOR PREVENTION OF SKIN AGING

(71) Applicant: Youna Son, Seoul (KR)

(72) Inventor: Youna Son, Seoul (KR)

(21) Appl. No.: 15/877,814

(22) Filed: Jan. 23, 2018

(30) Foreign Application Priority Data
Jan. 25, 2017 (KR) 10-2017-0012122
Jul. 20, 2017 (KR) 10-2017-0091989

(51) Int. Cl. A61K 8/9789; A61K 8/9717; A61K 8/66; A61Q 19/08; A61K 8/42; A61K 8/49; A61K 8/44; A61K 8/67; A61K 8/19; A61N 5/06

(52) U.S. Cl. CPC A61K 8/9789; A61K 8/9717; A61K 8/66; A61Q 19/08; A61K 8/42; A61K 8/4926; A61N 2005/067; A61K 8/676; A61K 8/19; A61N 5/062; A61N 5/0616; A61K 2800/81; A61K 8/44

(57) ABSTRACT

A kit and an aesthetic system for the prevention of skin aging includes a telomerase activating composition, a skin dermis irradiating laser, a composition for topical fat removal including a hyaluronidase, and a skin muscle layer irradiating laser, as well as a process for preventing skin aging in humans using said kit and system. The kit and aesthetic system for the prevention of skin aging have comprehensive effects concerning skin aging including instantaneous wrinkle improvement, skin contouring, elasticity enhancement, wrinkle improvement, topical fat removal, and skin hydration improvement as well as long-lasting maintenance effects up to 1 year to 1.5 years, despite their short treatment time.

### CROSS-REFERENCE TO RELATED APPLICATION

This application claims the benefit of Korean Patent Application Nos. 10-2017-0012122 and 10-2017-0091989, filed on Jan. 25, 2017 and Jul. 20, 2017, in the Korean Patent Office, the disclosures of which are incorporated herein in their entirety by reference.

### TECHNICAL FIELD

The present invention relates to a kit and an aesthetic system for the prevention of skin aging comprising a telomerase activating composition, a skin dermis irradiating laser, a composition for topical fat removal comprising hyaluronidase, and a skin muscle layer irradiating laser, as well as an aesthetic process for preventing skin aging in humans using said kit and system.

### BACKGROUND

Human skin constantly undergoes change over time, and representative of such change is the functional and aesthetic deterioration of the skin due to aging. In addition to aging occurring naturally in the body, various environmental factors trigger aging. Skin aging is generally classified into intrinsic aging resulting from genetic factors and extrinsic aging caused by external environmental factors such as sunlight.

Extrinsic skin aging results in the formation of wrinkles on the skin. Typical factors causing wrinkling include accumulation of fat, loss of subcutaneous muscles, reactive oxygen species, UV light, reduction of collagen biosynthesis, etc. That is, external physical or chemical factors, for example, heat, intense UV, physical abrasion (injury), acidic substances, etc. cause the destruction of skin tissues, which in turn causes the destruction of underlying collagen or elastin structures, gelatin, or the like, leading to the formation of wrinkles.

Specifically, collagen is a major matrix protein produced by fibroblasts in the skin and found in the extracellular matrix, is an important protein that accounts for 30% of the total body protein weight, and has a robust triple helical structure. Major functions of collagen have been reported to include maintenance of the mechanical firmness of the skin, the cohesion and resistance of the connective tissue, maintenance of the adhesion of cells, and inducement of cellular division and differentiation. Collagen is known to degrade in proportion to the accumulated exposure time to UV light. Thus, external factors such as UV light lead to the accumulation of elastic material and denaturation of collagen fibers in the skin dermis, resulting in the formation of wrinkles and reduction of elasticity in the skin.

Whereas intrinsic skin aging is difficult to modulate by artificial means, extrinsic skin aging is easier to control, compared to intrinsic skin aging, e.g., by the removal of reactive oxygen species, proliferation of fibroblasts, and stimulation of collagen biosynthesis. As research on the prevention and treatment of wrinkles develops, important functions of collagen in the skin are being identified. Studies show that when collagen metabolism increases by the stimulation of collagen synthesis in the skin, dermal matrix components increase, resulting in such effects as wrinkle improvement, enhanced elasticity, firmer skin, etc. Newly emerging wrinkle treatments include ultrasound treatment, skin scaling, laser peel, botulinum toxin injection, or the like. However, they are still unsatisfactory in terms of treatment effect and duration.

Genetically determined intrinsic skin aging can occur as a result of chromosomal damage or loss in skin cells. In particular, telomeres at the ends of chromosomes function to buffer the loss of genetic information in the form of shortening of DNA due to the DNA replication mechanism for cell division. With each round of cell division, chromosomes lose a certain length of DNA. Upon exceeding a certain number of cell divisions, telomeres become too shortened, eventually leading to the termination of cell division followed by cell death. On the other hand, it has been reported that cells have a prolonged life span if telomeres are kept long, which can be done by the enzyme telomerase. Thus, research on telomerases is ongoing as a strategy to prevent the genetic aging of cells.

### PRIOR ART

### Patent Literature

(Patent Literature 1) Korean Patent Publication No. 10-2014-0146074 (Dec. 24, 2014).

### SUMMARY OF THE INVENTION

The present invention is intended to address the above-mentioned problems. It is an object of the present invention to provide a kit and an aesthetic system for the prevention of skin aging utilizing the stimulation of collagen formation, stimulation of telomerase activity, recovery from loss of elasticity due to skin aging, wrinkle improvement, degradation of undesired fat layers, and skin contouring, as well as an aesthetic process for preventing skin aging in humans using said kit and system, for the comprehensive prevention of skin aging that can prevent or delay extrinsic skin aging as well as intrinsic skin aging. It is also an object of the present invention to provide a kit and an aesthetic system for the prevention of skin aging which have shorter treatment times, few complications after treatment, instantaneous wrinkle improvement and skin contouring effect, and long-lasting treatment effects, as well as an aesthetic process for preventing skin aging in humans using said kit and system.

The present inventor endeavored to develop a system for the prevention of skin aging by utilizing the stimulation of collagen formation, stimulation of telomerase activity, recovery from loss of elasticity due to skin aging, wrinkle improvement, degradation of undesired fat layers, and skin contouring and has discovered that the kit and aesthetic system for the prevention of skin aging of the present invention comprising a telomerase activating composition, a skin dermis irradiating laser, a composition for topical fat removal comprising hyaluronidase, and a skin muscle layer irradiating laser has the effect of comprehensive prevention of skin aging that can prevent or delay extrinsic skin aging as well as intrinsic skin aging.

The present invention comprises compositions and light sources (lasers) with certain frequencies that prevent skin aging by comprehensively preventing intrinsic skin aging due to genetic factors and extrinsic skin aging due to various factors and rejuvenating aged skin.

In the present invention, the prevention of skin aging includes stimulating collagen production, inducing collagen contraction and remodeling, contouring of sagging skin resulting from the loss of elasticity and accumulation of fat, enhancing skin elasticity, and improving wrinkles. The prevention of skin aging also includes removing or reducing undesirably accumulated topical fat, lipolysis in adipose cells, reducing edema, preventing or delaying intrinsic skin aging by activating telomerases in skin cells. The prevention of skin aging further includes improving skin hydration, improving skin roughness, and improving skin tone by improving pigmentation. That is, the prevention of skin aging in the present invention refers to, but is not limited to, improving or preventing one or more of the skin aging symptoms listed above.

In the following, A) a telomerase activating composition, B) a skin dermis irradiating laser, C) a composition for topical fat removal, and D) a skin muscle layer irradiating laser consisting the kit and aesthetic system for the prevention of skin aging according to the present invention, as well as treatment processes using the same will be explained in detail.

※A Telomerase Activating Composition (A)

As used herein, the term “telomerase” refers to an enzyme that maintains the length of telomeres, the genetic material made of repetitive nucleotide sequences found at the ends of chromosomes, thereby serving to prevent cellular senescence. Telomeres protect chromosomes from damage or fusion with other chromosomes, and their length shortens with each round of cell division. Upon exceeding a certain number of cell divisions, telomeres become too short, leading to the termination of cell division followed by cell death.

Thus, telomerase is an enzyme that serves to maintain cells at the pre-senescence stage by preventing the loss of cellular genetic material and genetic information. In the present invention, cellular senescence is prevented by using a telomerase activating composition that activates telomerase (increases the expression of telosome proteins) and keeps the telomere length long.

In the present invention, any composition that can activate telomerases may be used as the telomerase activating composition. The telomerase activating composition of the present invention is characterized in that it increases the expression of telosome subunit proteins in skin cells and/or maintains the telomere length of chromosomes in skin cells.

In the present invention, the telosome subunit protein refers to one or more selected from TRF1 (Telomeric Repeat Factor 1), TRF2 (Telomeric Repeat Factor 2), RAP1 (Repressor Activator Protein 1), TIN2 (TERF1-interacting nuclear factor 2), POT1 (Protection of telomeres 1), and TPP1 (Tripeptidyl peptidase 1).

In one embodiment, the telomerase activating composition of the present invention comprises Dendropanax morbifera Lev. extract, Kappaphycus alvarezii extract, or their mixture as the active ingredient. The Dendropanax morbifera Lev. extract or Kappaphycus alvarezii extract of the present invention can be prepared by grinding Dendropanax morbifera Lev. bark or Kappaphycus alvarezii followed by precipitation in a solvent (water, C₁ to C₆ alcohol or organic solvent such as ethanol, methanol, hexane, etc.) and treatment with a hydrolytic enzyme to obtain the extract, but are not limited thereto.

In the present invention, a telomerase activating composition may contain the active ingredient Dendropanax morbifera Lev. extract or Kappaphycus alvarezii extract in an amount of 0.00001 to 10% (w/w), 0.0001 to 5% (w/w), or 0.001 to 3% (w/w).

In the present invention, a telomerase activating composition can be formulated using conventional carriers, diluents or excipients such as lubricants, humectants, flavors, emulsifiers, suspending agents, preservatives, surfactants, etc. In one embodiment, the telomerase activating composition is a topical preparation. Specifically, it can be formulated as an essence, treatment, serum, emulsion, cream, ampule, lotion or pack, but is not limited thereto. According to its formulation, the above composition may include, in addition to the active ingredient Dendropanax morbifera Lev. extract or Kappaphycus alvarezii extract, anti-oxidants, stabilizers, solubilizers, vitamins, pigments, fragrances or the like that are commonly used in respective formulations.

Examples of said carriers, diluents and excipients include, but are not limited to, lactose, dextrose, sucrose, sorbitol, mannitol, starch, acacia gum, alginate, gelatin, calcium silicate, polyvinylpyrrolidone, cellulose, water, syrup, methyl hydroxybenzoate, propyl hydroxybenzoate, xanthan gum, talc, magnesium stearate, mineral oil, etc.

In another embodiment, the telomerase activating composition of the present invention is a cosmetic composition, and can further comprise substances that are well known in the field of cosmetic compositions to have a useful effect on the skin. For example, Viscum album extract, Imperata cylindrica root extract, soybean extract, and the like can be additionally included. Individual extracts of the present invention may be extracts fermented by microorganisms such as yeast or lactobacillus, but are not limited thereto.

As a preparation example for the telomerase activating composition of the present invention, an ampule and a cream containing 0.00056% (w/w) of Kappaphycus alvarezii extract were prepared (Table 1 and Table 2).

The amount of the telomerase activating composition of the present invention to be applied to the skin varies according to the skin condition, formulation of the composition, time of application, or the like, but can be suitably selected by a person skilled in the art. Specifically, a single dose of the composition is 3 to 20 cc, and the composition can be applied 1 to 5 times daily as needed.

In the present invention, application of the telomerase activating composition may be accompanied by a treatment with an ultrasound laser having a frequency of 10 to 90 Hz. Here, accompanying treatment means that the treatment with an ultrasound laser is carried out simultaneously with or immediately after the application of the telomerase activating composition, with the irradiation lasting for 5 to 20 minutes. In one specific embodiment of the present invention, an ultrasound laser having a frequency of 30 Hz is irradiated for 10 minutes after 10 cc of a telomerase activating composition is applied to the skin.

※Skin Dermis Irradiating Laser (B)

The skin dermis irradiating laser of the present invention is a laser having a frequency of 2 to 100 Hz or an RF (Radio Frequency) laser, and delivers heat energy to the dermis or to the dermis and SMAS (Superficial Musculoaponeurotic System). The skin dermis irradiating laser of the present invention may be one or more lasers. For example, it is one or more lasers selected from the group consisting of [a laser having a frequency of 2 to 9 Hz], [a laser having a frequency of 30 to 50 Hz] and [an RF (35 MHz to 45 MHz) laser].

A skin dermis irradiating laser having a wavelength of 1064 nm and a frequency of 2 to 9 Hz, used in the present invention, penetrates deep into the dermis and delivers heat energy, thereby stimulating collagen production. The irradiation time can be, but is not limited to, 1 to 10 minutes.

The skin dermis irradiating laser having a frequency of 30 to 50 Hz, used in the present invention, works to deliver the laser energy (heat energy of 65 r) to the deep dermis and SMAS using high intensity focused ultrasound (HIFU). This causes contraction of the muscle layer under the dermis, resulting in the reduction of wrinkles, and helps the production of collagen and elastin. At the same time, this laser helps lipolysis if there are excessive fat layers. This laser can be operated in two different modes according to the depth reached by the laser wave. In one mode, the laser wave reaches 3.5 mm deep under the skin surface and contracts the dermal layers, thereby reducing wrinkles and stimulating collagen and elastin production. In the other mode, the laser wave reaches 4.5 mm deep under the skin surface, helping the lipolysis of fat layers in the skin. Treatments can include either one of the above two modes, or both modes. The irradiation time can be 10 to 40 minutes respectively, but is not limited thereto.

A 92 W RF laser (35 MHz to 45 MHz) used in the present invention allows the RF wave to penetrate the skin dermis/fat layers, generating heat deep in the dermis and thereby inducing tissue contraction and reorganization. As the laser generates heat in the connective tissue, blood flow and circulation increase, resulting in tissue firming and improved skin texture. It is also an effect of this laser that intercellular water and noxious substances are discharged, resulting in a visible slimming of the skin contour in the irradiated area. This laser can be irradiated for 3 to 10 minutes in the unipolar mode, but is not limited thereto.

The laser of the present invention to deliver energy to the deep dermis can be selected from the above three types of laser. It may be any combination selected from the above three types of laser, or all three types of laser may be used together.

In one specific example of the present invention for the treatment with a skin dermis irradiating laser, a laser with a wavelength of 1064 nm and a frequency of 5 Hz (GentleMax) was irradiated for 3 or 5 minutes, and then Indigo laser with a frequency of 30 to 50 Hz was irradiated in mode A (laser wave reaches 3.5 mm deep under the skin surface) and mode B (laser wave reaches 4.5 mm deep under the skin surface) for 10 or 20 minutes, respectively. Finally, the skin was irradiated with an RF laser having a frequency of 40.68 MHz for 6 minutes.

By delivering heat energy to the dermis or to the dermis and SMAS, the skin dermis irradiating laser of the present invention can bring about one or more of the following changes:
i) contraction and reorganization of the dermis tissue;
ii) contraction of the skin muscle layer;
iii) induction of collagen, elastin, or collagen and elastin production;
iv) increases in the blood flow and lymphatic circulation in the skin connective tissue; and
v) discharge of water and noxious substances present in the skin.

※A Composition for Topical Fat Removal (C)

The composition for topical fat removal of the present invention comprises 300 IU to 600 IU of hyaluronidase per single dose volume, and may further comprise one or more of a local anesthetic, an antihistamine, a lipolysis stimulator and a collagen production stimulator.

Hyaluronidase is a water-soluble enzyme secreted from a mammalian vas deferens or testes, characterized by its actions to remove barriers between tissues by hydrolyzing the glucosaminic bonds between hyaluronic acid, a major intercellular substance, and connective tissues to dissolve the bonds and reduce fibroplasia in tissues. Hyaluronidase is also known to relieve swelling and edema in tissues.

Utilizing the characteristics of hyaluronidase that hydrolyzes and dissolves substances within skin tissues, LLD (Lipolytic Lymph Drainage) treatment is widely used in which hyaluronidase is directly injected subcutaneously to degrade substances including fibers in subcutaneous tissues, thereby facilitating lymphatic circulation to help drainage of adipose tissue to reduce adipose tissue and relieve obesity. However, the above treatment, though helpful in reducing adipose tissue, has been associated with various side effects since it uses high doses (2000 to 15000 IU) of hyaluronidase.

Existing LLD treatments using high doses of hyaluronidase cause allergic symptoms such as redness or itching, and excessive lysis of adipose tissue leads to bruising due to subcutaneous bleeding or dimpling. LLD treatments have drawbacks that treatment has to be discontinued frequently to take care of such side effects and continuous treatment is difficult due to patients' repulsion due to pain.

Furthermore, using high doses of hyaluronidase makes it difficult to reduce the interval between injection points (about 5-cm interval). As a result, fat removal is concentrated only around injection points, causing irregularities such as indentations, sagging and wrinkling in the skin as well as substantial reduction in skin elasticity and consequently resulting in the dissatisfaction of patients.

Having endeavored to address the above problem, the present inventor discovered that the minimum amount of hyaluronidase exhibiting efficacy is 300 IU and that above 600 IU, adipose cells are excessively hydrolyzed, causing concerns about dimpling or subcutaneous bleeding. Thus, by adjusting the single dose of hyaluronidase in the injection composition for topical fat removal of the present invention to 300 IU to 600 IU, the side effects from using the drug are minimized and yet fat removal over a wide area can be carried out using a minimal volume.

It has been shown in clinical trials that at a hyaluronidase activity of 300 IU and an injection volume of 1 cc, the diameter of the diffusion range for the injection composition is about 1 cm. Thus, by injecting the composition at narrow intervals between injection points of 0.5 to 2 cm, even fat removal as well as collagen production by skin stimulation can be facilitated.

Most preferably, the injection composition of the present invention is injected to give 300 IU to 600 IU of hyaluronidase per injection, at a single dose volume of 0.5 to 2 cc with an interval between injection points of 0.5 to 1.5 cm. The daily dose of hyaluronidase is preferably 15,000 IU or less, and the single dose volume and injection points can be suitably adjusted according to the IU of hyaluronidase.

The injection composition of the present invention can be injected 50 times on a fixed interval based on an injection volume of 1 cc containing 300 IU of hyaluronidase, and the site of injection can be any body area where fat is accumulated including abdomen, buttocks, thighs, calves, chin, forehead, arms, and the like.

The injection composition of the present invention may also comprise a local anesthetic to alleviate the pain from injection, an antihistamine to prevent allergic side effects, a lipolysis stimulator to rapidly transport fatty acids from degraded fat into mitochondria and degrade them, and a collagen stimulator. As a result, the injection composition prevents allergic symptoms like redness or itching as well as pain and bruising, accelerates fat removal, and excels in maintaining skin resilience by collagen formation.

For the above local anesthetic, lidocaine can be used in an amount of 0.08 to 0.4% by weight based on the total weight of the composition. When the amount of the local anesthetic is below 0.08%, analgesic effect is unsatisfactory, and when the amount is above 0.4%, the duration of anesthesia is prolonged and is uneconomical.

As for the other ingredients, the antihistamine Peniramin (pheniramine) is contained in an amount of 0.01 to 0.02% by weight, the lipolysis stimulator L-carnitine in an amount of 0.01 to 4.0% by weight, and the collagen synthesis stimulator vitamin C in an amount of 0.1 to 2.0% by weight.

The composition of the present invention may also comprise one or more substances selected from a tonicity agent, a nonionic surfactant, a stabilizer, a preservative, etc. that are commonly used in injectable solutions, within dose ranges allowable for injection compositions. The composition of the present invention may further comprise a lipolytic agent such as phosphatidylcholine, aminophylline, caffeine, or the like as well as placenta components or pentoxifylline which improves blood circulation, in amounts of 0.01 to 4.0% by weight.

The amounts of the above ingredients were determined within their respective maximum daily doses in preparation for a case where 300 IU of hyaluronidase is administered at the desirable daily dose of 15,000 IU.

In one embodiment for the injection composition for topical fat removal of the present invention, the injection composition comprises 300 IU to 600 IU of hyaluronidase per single dose volume and, based on the weight of the total composition, 0.08 to 0.4% by weight of lidocaine as local anesthetic, 0.01 to 0.02% by weight of Peniramin as antihistamine, 0.01 to 4.0% by weight of L-carnitine as lipolysis stimulator, and 0.1 to 2.0% by weight of vitamin C as collagen synthesis stimulator, with the remainder being saline solution, and the composition is injected at a volume of 0.5 to 2 cc per single dose with an interval between injection points of 0.5 to 1.5 cm.

In a specific preparation example of the injection composition for topical fat removal of the present invention, 100 cc of a composition comprising 30,000 IU to 60,000 IU of hyaluronidase, 210 mg of lidocaine, 12 mg of Peniramin, 660 mg of L-carnitine, and 600 mg of vitamin-C was prepared.

※A Skin Muscle Layer Irradiating Laser (D)

The skin muscle layer irradiating laser of the present invention is irradiated from inside the mouth toward the face and is a laser having a frequency of 1 to 3.5 Hz which delivers energy to the skin muscle layer. As this laser delivers energy to the muscle layer, it has excellent effects on collagen synthesis, wrinkle improvement, and elasticity enhancement. Particularly, it can effectively deliver energy to the skin muscle layer by irradiating the laser beam from inside the mouth toward the face.

Specifically, this laser treatment is painless, and provides a thermal effect to the oral mucosa in a non-ablative, non-invasive manner without anesthesia to give a remodeling and tightening effect. The intraoral treatment of this step applies a laser beam delivering controlled energy such that a temperature in the range of from 45 to 70° C., or from 45 to 65° C., is reached in the upper dermis, which can lead to an instantaneous contraction of 30% of the tissue. The instantaneous contraction of the upper dermis mechanically pulls up the underlying deeper tissue layers, creating a lifting effect.

Since the above laser treatment delivers energy through the oral mucosa, it can protect the epidermis and stimulate collagen remodeling and neo-collagenesis (lasting up to 6 months). In addition, this laser treatment has the advantage that it is accompanied by no bleeding or pain except some warmth felt in the oral mucosa and it does not affect the daily life after the treatment since oral mucosa is particularly quick to recover.

In one specific example of the present invention for the treatment with a skin muscle layer irradiating laser, a skin muscle layer irradiating laser with a frequency of 1.8 Hz or 3 Hz was irradiated from inside the mouth toward the face for 5 or 6 minutes.

By delivering heat energy to the skin muscle layer or to the skin muscle layer and upper dermis, the skin muscle layer irradiating laser of the present invention can bring about one or more of the following changes:
i) collagen remodeling and neo-collagenesis in the skin muscle layer;
ii) contraction of the skin muscle layer; and
iii) tissue contraction in the upper dermis.

Detailed embodiments of the present invention will be described in the following.

To achieve the objective of the present invention, the present invention provides in one aspect a kit for the prevention of skin aging, comprising A) a telomerase activating composition; B) a skin dermis irradiating laser; C) a composition for topical fat removal comprising 300 IU to 600 IU of hyaluronidase; and D) a skin muscle layer irradiating laser.

Also, a kit for the prevention of skin aging according to the present invention may further comprise an ultrasound laser having a frequency of 10 to 90 Hz that is applied simultaneously with or immediately after the application of the telomerase activating composition.

In the kit of the present invention, treatments with the above elements A) to D) may be carried out irrespective of their order, e.g., in the order stated, in an inverse order, or in a random order.

For example, the kit of the present invention may be used in the order of [A), B), C) and D)], [A), B), D) and C)], [A), C), B), and D)], [A), C), D), and B)], [A), D), B), and C)], [A), D), C), and B)], [B), A), C), and D)], [B), A), D), and C)], [B), C), A), and D)], [B), D), A), and C)], [B), D), C), and A)], [C), A), B), and D)], [C), A), D), and B)], [C), B), A), and D)], [C), B), D), and A)], [C), D), A), and B)], [C), D), B), and A)], [D), A), B), and C)], [D), A), C), and B)], [D), B), A), and C)], [D), B), C), and A)], [D), C), A), and B)], or [D), C), B), and A)].

In addition, the kit of the present invention may be repeatedly used in 1 to 10 cycles, but is not limited thereto. In each cycle, treatments with the above elements A) to D) may be carried out irrespective of their order.

In another aspect, the present invention provides an aesthetic system for the prevention of skin aging, comprising A) a telomerase activating composition; B) a skin dermis irradiating laser; C) a composition for topical fat removal comprising 300 IU to 600 IU of hyaluronidase; and D) a skin muscle layer irradiating laser.

The telomerase, the telomerase activating composition, the skin dermis irradiating laser, hyaluronidase, the composition for topical fat removal comprising 300 IU to 600 IU of hyaluronidase, and the skin muscle layer irradiating laser of the present invention are as described above.

The aesthetic system according to the present invention may further comprise an ultrasound laser having a frequency of 10 to 90 Hz that is applied simultaneously with or immediately after the application of the telomerase activating composition.

The aesthetic system of the present invention comprises the following elements. When using the aesthetic system, treatments with the following elements may be carried out irrespective of their order, e.g., in the order stated, in an inverse order, or in a random order:
i) a telomerase activating composition to be applied to the skin;
ii) a skin dermis irradiating laser to be irradiated on the skin for 3 to 60 minutes;
iii) a composition for topical fat removal to be injected at a single dose volume of 0.5 to 2 cc with an interval between injection points of 0.5 to 1.5 cm; and
iv) a skin muscle layer irradiating laser to be irradiated from inside the mouth toward the face for 3 to 10 minutes.

In addition, the aesthetic system of the present invention may further comprise irradiating an ultrasound laser having a frequency of 10 to 90 Hz for 5 to 20 minutes simultaneously with or immediately after the treatment with element i).

For example, the aesthetic system of the present invention may be used in the order of [i), ii), iii), and iv)], [i), ii), iv), and iii)], [i), iii), ii), and iv)], [i), iii), iv), and ii)], [i), iv), ii), and iii)], [i), iv), iii), and ii)], [ii), i), iii), and iv)], [ii), i), iv), and iii)], [ii), iii), i), and iv)], [ii), iv), i), and iii)], [ii), iv), iii), and i)], [iii), i), ii), and iv)], [iii), i), iv), and ii)], [iii), ii), i), and iv)], [iii), ii), iv), and i)], [iii), iv), i), and ii)], [iii), iv), ii), and i)], [iv), i), ii), and iii)], [iv), i), iii), and ii)], [iv), ii), i), and iii)], [iv), ii), iii), and i)], [iv), iii), i), and ii)], or [iv), iii), ii), and i)].

In the aesthetic system of the present invention, the skin dermis irradiating laser in ii) above is one or more lasers selected from the group consisting of a laser having a frequency of 5 Hz, a laser having a frequency of 30 to 50 Hz and an RF laser. The laser having a frequency of 5 Hz may be irradiated for 3 to 8 minutes, and the laser having a frequency of 30 to 50 Hz for 15 to 40 minutes. The RF laser may be irradiated for 4 to 10 minutes with a frequency of 40.68 MHz.

In addition, the aesthetic system of the present invention may be repeatedly used in 1 to 10 cycles, but is not limited thereto. In each cycle, treatments with the above elements i) to iv) may be carried out irrespective of their order. For example, treatments with elements i), ii), iii), and iv) may be carried out in the order stated with an interval of one week, and with this entire process considered as one cycle, the treatments can be repeated for three cycles.

In one specific treatment example of the present invention, a telomerase activating composition containing 0.00056% (w/w) of Kappaphycus alvarezii extract was applied, followed by irradiation with an ultrasound laser having a frequency of 30 Hz for 10 minutes. Next, as skin dermis irradiating lasers, a laser having a frequency of 5 Hz (GentleMax) was irradiated for 5 minutes, and Indigo laser, a laser having a frequency of 30 to 50 Hz, was irradiated in mode A and mode B for 20 minutes respectively, followed by irradiation with an RF laser with a frequency of 40.68 MHz for 6 minutes. Then, using 100 cc of a composition for topical fat removal comprising 30,000 IU to 60,000 IU of hyaluronidase, 210 mg of lidocaine, 12 mg of Peniramin, 660 mg of L-carnitine, and 600 mg of vitamin-C, a single dose volume of 1 cc each was injected at 50 points with an interval between injection points of 1 cm, four times with an interval of one week. Thereafter, a skin muscle layer irradiating laser having a frequency of 1.8 Hz or 3 Hz was irradiated from inside the mouth toward the face for 5 minutes.

In another treatment example of the present invention, 3 cc of a telomerase activating composition containing 0.00056% (w/w) of Kappaphycus alvarezii extract was applied, followed by irradiation with an ultrasound laser having a frequency of 30 Hz for 10 minutes. Next, as skin dermis irradiating lasers, a laser having a frequency of 5 Hz (GentleMax) was irradiated for 3 minutes, and Indigo laser, a laser having a frequency of 30 to 50 Hz, was irradiated in mode A and mode B for 10 minutes respectively, followed by irradiation with an 90 w RF laser in the unipolar mode for 6 minutes. Then, 100 cc of a composition for topical fat removal comprising 30,000 IU to 60,000 IU of hyaluronidase, 210 mg of lidocaine, 12 mg of Peniramin, 660 mg of L-carnitine, and 600 mg of vitamin-C was prepared and a single dose volume of 1 cc each was injected at 10 points with an interval between injection points of 1 cm. Thereafter, a skin muscle layer irradiating laser having a frequency of 3 Hz was irradiated from inside the mouth toward the face for 6 minutes.

In another aspect, the present invention provides an aesthetic process for preventing skin aging using the kit and aesthetic system for the prevention of skin aging according to the present invention.

The kit and aesthetic system for the prevention of skin aging according to the present invention are as described above.

Specifically, the present invention provides an aesthetic process for the prevention of skin aging, wherein the following steps are carried out at least once irrespective of their order:
i) a step of applying a telomerase activating composition;
ii) a step of irradiating a skin dermis irradiating laser for 3 to 60 minutes;
iii) a step of injecting a composition for topical fat removal at a single dose volume of 0.5 to 2 cc with an interval between injection points of 0.5 to 1.5 cm; and
iv) a step of irradiating a skin muscle layer irradiating laser from inside the mouth toward the face for 3 to 10 minutes.

In addition, the aesthetic process of the present invention may further comprise irradiating an ultrasound laser having a frequency of 10 to 90 Hz for 5 to 20 minutes simultaneously with or immediately after step i).

In the present invention, the above steps may be carried out irrespective of their order, e.g., in the order stated, in an inverse order, or in a random order. For example, the above steps may be carried out in the order of [i), ii), iii), and iv)], [i), ii), iv), and iii)], [i), iii), ii), and iv)], [i), iii), iv), and ii)], [i), iv), ii), and iii)], [i), iv), iii), and ii)], [ii), i), iii), and iv)], [ii), i), iv), and iii)], [ii), iii), i), and iv)], [ii), iv), i), and iii)], [ii), iv), iii), and i)], [iii), i), ii), and iv)], [iii), i), iv), and ii)], [iii), ii), i), and iv)], [iii), ii), iv), and i)], [iii), iv), i), and ii)], [iii), iv), ii), and i)], [iv), i), ii), and iii)], [iv), i), iii), and ii)], [iv), ii), i), and iii)], [iv), ii), iii), and i)], [iv), iii), i), and ii)], or [iv), iii), ii), and i)].

The above steps i) to iv) may be carried out by the same person, and suitable time intervals may be placed between the steps. The suitable time interval between the steps may be 1 to 60 minutes, 1 to 24 hours, or 1 day to 2 weeks, depending on the skin condition and the preparation time for the next treatment. The time interval can be determined by those skilled in the art and may vary according to the treatment step and skin condition.

The skin dermis irradiating laser in step ii) of the present invention is one or more lasers selected from the group consisting of a laser having a frequency of 5 Hz, a laser having a frequency of 30 to 50 Hz and an RF laser. The laser having a frequency of 5 Hz may be irradiated for 3 to 8 minutes, and the laser having a frequency of 30 to 50 Hz for 15 to 40 minutes. The RF laser may be irradiated for 4 to 10 minutes with a frequency of 40.68 MHz

The kit and aesthetic system for the prevention of skin aging according to the present invention have comprehensive effects concerning skin aging including instantaneous wrinkle improvement, skin contouring, elasticity enhancement, wrinkle improvement, topical fat removal, and skin hydration improvement as well as long-lasting maintenance effects up to 1 year to 1.5 years, despite their short treatment time. Thus, the present invention has excellent effects in terms of preventing skin aging.

### BRIEF DESCRIPTION OF THE DRAWINGS

FIG. 1 illustrates the expression of the telosome proteins POT1 and TPP1 in fibroblasts treated with 0.25% or 0.5% Kappaphycus alvarezii extract after induction of senescence with H₂O₂.

FIG. 2 illustrates the telomere length in fibroblasts treated with 0.25% or 0.5% Kappaphycus alvarezii extract after induction of senescence with H₂O₂.

FIG. 3 shows photographs illustrating the extent of senescence determined using a (3-galactosidase staining kit (Sigma Senescent cell staining kit) in fibroblasts treated with 0.25% or 0.5% Kappaphycus alvarezii extract after induction of senescence with H₂O₂.

FIG. 4 shows the extent of senescence measured by a confocal microscope on the starting day and Day 42 in a clinical study to verify the preventive effect of a composition comprising Kappaphycus alvarezii extract on skin aging.

FIG. 5 shows the extent of senescence measured by a 3D scanner on the starting day and Day 42 in a clinical study to verify the preventive effect of a composition comprising Kappaphycus alvarezii extract on skin aging.

FIG. 6 shows the results of the perception tests to gauge the age of subjects on the starting day and Day 42 in a clinical study to verify the preventive effect of a composition comprising Kappaphycus alvarezii extract on skin aging.

FIG. 7 is photographs of skin contours showing enhanced elasticity in the double chin and cheek areas immediately after irradiation with a skin dermis irradiating laser.

FIG. 8 is photographs of nasolabial wrinkles after 1 session of treatment with a skin muscle layer irradiating laser.

FIG. 9 shows the number of collagen fibers measured in skin biopsies taken from 90 subjects grouped into a test group and Controls 1 to 8 at baseline as well as 1 week, 2 weeks, 3 weeks and 4 weeks post-treatment to comprehensively investigate the effects of the kit and aesthetic system for the prevention or treatment of skin aging according to the present invention. The test group, representing the present invention, was treated with A) a telomerase activating composition, B) a skin dermis irradiating laser, C) a composition for topical fat removal, and D) a skin muscle layer irradiating laser, while the control groups received only some of the treatments. Specifically, Control 1 received treatment A; Control 2 treatment B; Control 3 treatment C; Control 4 treatment D; Control 5 treatments A, B, and C; Control 6 treatments A, B, and D; Control 7 treatments A, C, and D; Control 8 treatments B, C, and D.

FIG. 10 shows the skin elasticity improvement measured in the subjects in the test group and Controls 1 to 8 at baseline as well as 1 week, 2 weeks, 3 weeks and 4 weeks post-treatment. A DermaLab USB elasticity probe (Cortex Technology, Inc.) was employed to assess the changes in skin elasticity.

FIG. 11 shows the wrinkle improvement effect determined by measuring the wrinkling in the subjects in the test group and Controls 1 to 8 at baseline as well as 1 week, 2 weeks, 3 weeks and 4 weeks post-treatment. A device quantifying the extent of wrinkling (Aramo TS, programmed by Aram HUVIS Co., South Korea) was used to assess the changes in wrinkling.

FIG. 12 shows the facial contouring effect measured in the subjects in the test group and Controls 1 to 8 at baseline as well as 1 week, 2 weeks, 3 weeks and 4 weeks post-treatment. A SuperSonic Imagine (Les Jardins de la Duranne, Aix en Provence, France) ultrasound, which measures the thickness of subcutaneous fat in the face area, was used for the assessment.

FIG. 13 shows the telomerase activating effect measured in the subjects in the test group and Controls 1 to 8 at baseline as well as 1 week, 2 weeks, 3 weeks and 4 weeks post-treatment. Blood samples from the subjects were analyzed by sandwich enzyme immunoassay to measure the amount of telomerase.

FIG. 14 shows the skin hydration improvement measured in the subjects in the test group and Controls 1 to 8 at baseline as well as 1 week, 2 weeks, 3 weeks and 4 weeks post-treatment. Skin hydration was determined by measuring the capacitance reflecting the water content, which has the highest dielectric constant in the skin.

FIG. 15 shows the skin roughness improvement determined by measuring skin roughness in the subjects in the test group and Controls 1 to 8 at baseline as well as 1 week, 2 weeks, 3 weeks and 4 weeks post-treatment. Images of the test areas were taken using a Beauty scope (×60 lens) with a diameter of 30 mm, and the skin surface contour was transformed into a graph and numbers to obtain skin roughness values.

FIG. 16 shows the changes in the amount of pigmentation measured in the subjects in the test group and Controls 1 to 8 at baseline as well as 1 week, 2 weeks, 3 weeks and 4 weeks post-treatment. A RSA (Robo Skin Analyzer CS 50, In Forward. Inc., Japan) allows for a combined examination of pores, pigmentation, wrinkles, skin tone, etc., and the instrument was used to measure changes in the amount of pigmentation.

### DETAILED DESCRIPTION

The following Reference Examples and Examples illustrate how the present inventor developed a kit and aesthetic system for the prevention of skin aging, as well as an aesthetic process for preventing skin aging in humans using said kit and system according to the present invention.

### REFERENCE EXAMPLES

### Reference Example 1: Preparation of a Telomerase Activating Extract and Verification of its Effect

(1) Preparation of a Telomerase Activating Extract

A Kappaphycus alvarezii extract was used to prepare a telomerase activating composition. Specifically, Kappaphycus alvarezii was ground and then precipitated in a solvent (water, C₁ to C₆ alcohol or organic solvent such as ethanol, methanol, hexane, etc.), followed by the addition of a hydrolytic enzyme to obtain an extract.

(2) In Vitro Experiment on the Prevention of Skin Aging

The in vitro preventive effect of the Kappaphycus alvarezii extract prepared above on skin aging was investigated. When telomerases are activated, the length of telomeres at the ends of chromosomes in the cells is maintained or lengthened, prolonging the life span of cells. In this regard, an experiment on the senescence of human fibroblasts was carried out.

A human fibroblast cell line was placed in a 100-mm plate (Corning 100 mm TC-Treated Culture Dish, Product #430167) at a concentration of 1800 cells/cm² and treated with a fibroblast medium containing a Kappaphycus alvarezii extract at a final concentration of 0.25% or 0.5%. Then, senescence was induced by treating with H₂O₂ (hydrogen peroxide 4×40 uM) for 96 hours. Using pre-senescent fibroblasts and fibroblasts aged by induction of senescence using the same method but without a treatment with a Kappaphycus alvarezii extract as controls, the length of telomeres and the expression of telosome proteins in the groups were compared.

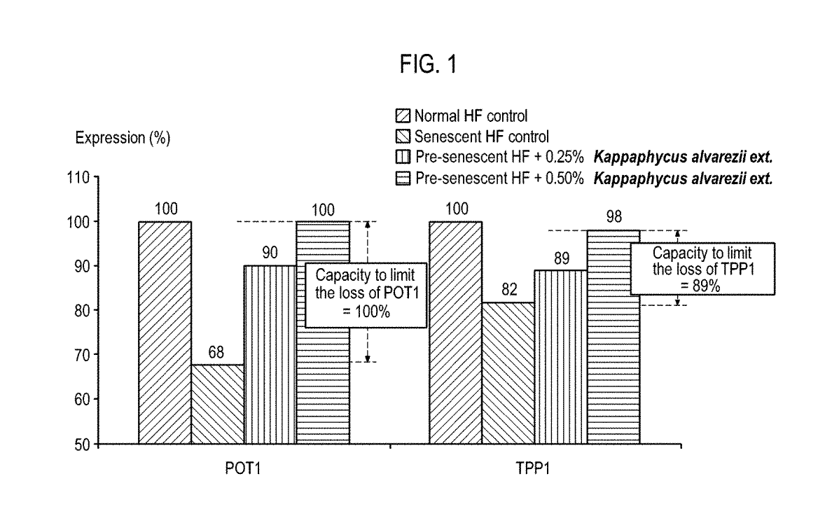

The expression of POT1 and TPP1, telosome proteins working to protect and maintain the length of telomeres, was maintained high in the test groups treated with a Kappaphycus alvarezii extract. Particularly, expression of the above proteins was maintained at a level close to that in normal pre-senescent fibroblasts when the cells were treated with a Kappaphycus alvarezii extract at a concentration of 0.5%. In contrast, the expression of the above two proteins was reduced to 68% and 82%, respectively, in fibroblasts aged by induction of senescence without a treatment with a Kappaphycus alvarezii extract (FIG. 1).

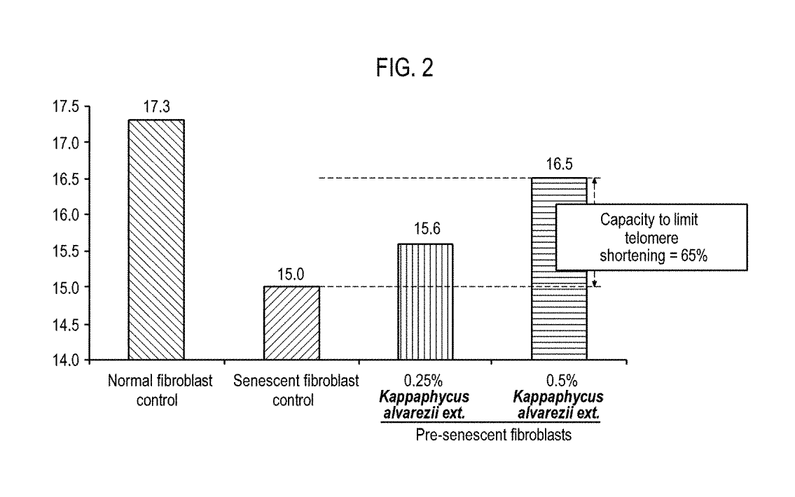

In addition, telomere shortening results showed that treatment with a Kappaphycus alvarezii extract at a concentration of 0.5% could inhibit about 65% of telomere shortening resulting from senescence (FIG. 2).

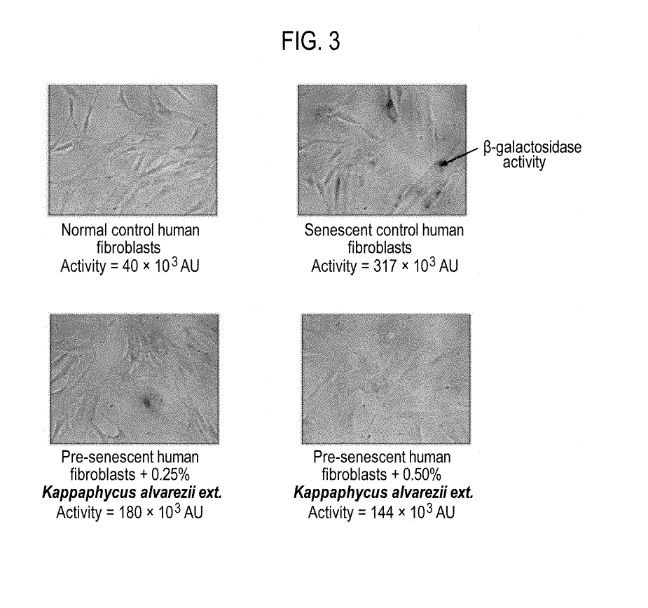

Lastly, the results from an experiment in which senescence was induced in fibroblasts under the same conditions as described above and the extent of senescence was determined using a (3-galactosidase staining kit (Sigma Senescent cell staining kit) showed that the extent of cellular senescence was significantly reduced in cells treated with a Kappaphycus alvarezii extract at a concentration of 0.5% (FIG. 3).

(3) In-Vivo Experiment on the Prevention of Skin Aging

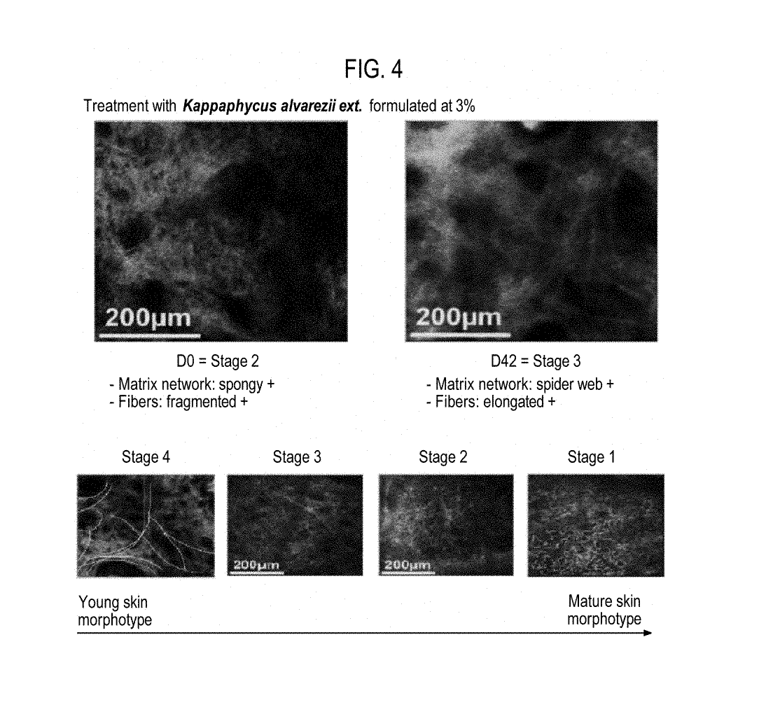

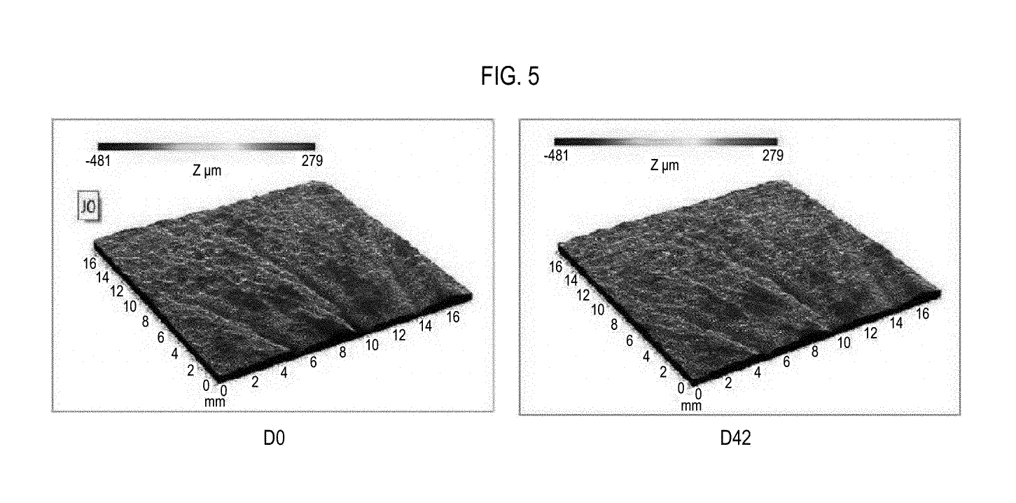

Healthy volunteers with an average age of 59±5 were divided into a placebo group of 21 subjects and a test group of 24 subjects. For the test group, an emulsion preparation comprising 3% of Kappaphycus alvarezii extract was applied to the subjects' skin in the entire facial area (2 times daily). Comparisons were made for the skin surface conditions measured on the day of starting the experiment (Day 0) and Day 42 using a confocal microscope (Vivascope 1500 Trilaser Lucid®-Mavig GmbH) and a 3D scanner (Fringe projection, Eotech, France; analyzed with Optocat software) (FIG. 4 and FIG. 5). In this regard, senescence Stage 1 denotes the most senescent state and Stage 4 refers to young skin with the least progressed senescence.

As a result, the test group showed an ameliorated extent of skin aging at Day 42 relative to Day 0 compared to the placebo group, as illustrated by FIG. 4. 58% of the subjects in the test group showed an improvement in their skin stage, and 46% of those subjects showed an improvement from senescence Stage 1 or 2 to Stage 3 or 4.

FIG. 5 illustrates the measurements of wrinkle depth in the ocular area, showing that the wrinkle depth improved 27.9% at Day 42 relative to Day 0.

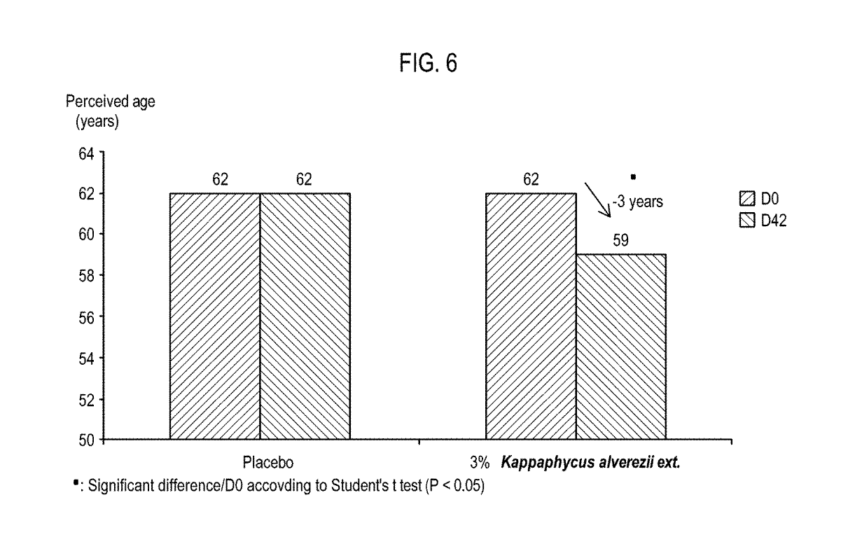

In a perception test to gauge the average age of the above subjects based on their photographs taken before and after the experiment, the average age of the placebo group was 62 years, with no difference between the before and after photos. In contrast, the test group subjects were perceived as appearing 3 to 6 years younger on average. 88% of the subjects in the test group were perceived as looking younger (FIG. 6).

### Reference Example 2: Accompanying Treatment with a Telomerase Activating Composition and an Ultrasound Laser

(1) Preparation of a Telomerase Activating Composition

The Kappaphycus alvarezii extract obtained in Reference Example 1 was used for the preparation of a telomerase activating composition. Specifically, a telomerase activating composition comprising the Kappaphycus alvarezii extract prepared in Reference Example 1 was formulated into a cream and an ampule having the composition shown below.

TABLE 1
Composition of the telomerase activating cream

| Ingredients | Content % (w/w) |
|---|---|
| Glycerin | 8.0 |
| Sunflower seed oil | 3.2 |
| Stearic acid | 2.5 |
| Mango seed butter | 1.5 |
| Kappaphycus alvarezii extract | 0.0056 |
| Yeast/Viscum album extract fermented | 0.007 |
| Lactobacillus/soybean extract fermented | 0.0036 |
| Yeast/Imperata cylindrica root extract | 0.0026 |
| fermented | |
| Purified water | q.s. |

TABLE 2
Composition of the telomerase activating ampule

| Ingredients | Content % (w/w) |
|---|---|
| Glycerin | 22.0 |
| Sodium PCA | 12.0 |
| Xanthan gum | 0.2 |
| Citric acid | 0.15 |
| Kappaphycus alvarezii extract | 0.0056 |
| Yeast/Viscum album extract fermented | 0.007 |
| Lactobacillus/soybean extract fermented | 0.0036 |
| Yeast/Imperata cylindrica root extract | 0.0026 |
| fermented | |
| Purified water | q.s. |

(2) Effect of the Accompanying Treatment with a Telomerase Activating Composition and an Ultrasound Laser

After the above telomerase activating composition in the form of a topical preparation was applied to the skin of the 24 test group subjects in Reference Example 1, an ultrasound laser with a frequency of 10 to 90 Hz was irradiated to facilitate the delivery of the composition into the skin.

Specifically, after 10 cc of the above telomerase activating composition in the form of a topical preparation was applied to the skin, a laser (Legato—Alma Lasers, Israel; and Sonopact—Medius, South Korea) set at a frequency of 30 Hz was irradiated for 10 minutes over the entire face area in 1 to 2 passes.

In this study, the ultrasound laser i) had a vibration effect (hammering-impact effect; push-pull effect) on the treated skin area, and ii) caused a thermal effect resulting from vibration in the epidermal tissue and the dermis. In addition, it iii) destroyed degenerated keratinocytes resulting from ultrasound application, and effectively delivered the composition to the dermis by facilitating the composition to penetrate into the intercellular spaces.

### Reference Example 3: Laser Irradiation of the Dermis and Collagen Production Stimulating Effect

In this experiment, a laser reaching the dermis was irradiated on the entire facial area and the chin area following the treatment described in Reference Example 2. In this step, a laser delivering energy deep into the dermis was used to stimulate collagen synthesis in the dermis.

Three kinds of laser were used for the laser irradiation of the dermis in the present Reference Example. First, a laser having a wavelength of 1064 nm and a frequency of 5 Hz (GentleMax—Candela, U.S.A.) was irradiated on the skin surface for 5 minutes so that the laser energy penetrates deep into the dermis to deliver heat energy.

Next, a laser having a frequency of 30 to 50 Hz (Indigo-K1MED, South Korea) delivered the laser energy (heat energy of 65° C.) to the deep dermis and SMAS using high intensity focused ultrasound (HIFU). This laser has mode A and mode B, and the treatment consisted of irradiation in mode A and mode B for 20 minutes each. This caused contraction of the muscle layer under the dermis, resulting in reduction of wrinkles and stimulation of collagen and elastin production. At the same time, this laser irradiation induced lipolysis if there are excessive fat layers at a depth of 10 to 20 mm.

Lastly, a 92 W RF laser (40.68 MHz; Tenor Laser-Alma lasers, Israel) was irradiated in the unipolar mode for 6 minutes. This allowed the RF wave to penetrate the skin dermis/fat layers and generate heat deep in the dermis, inducing tissue contraction and reorganization. As the laser generated heat in the connective tissue, blood flow and circulation increased, resulting in tissue firming and improved skin texture. Also, intercellular water and noxious substances were discharged, resulting in a visible slimming of the skin contour in the irradiated area.

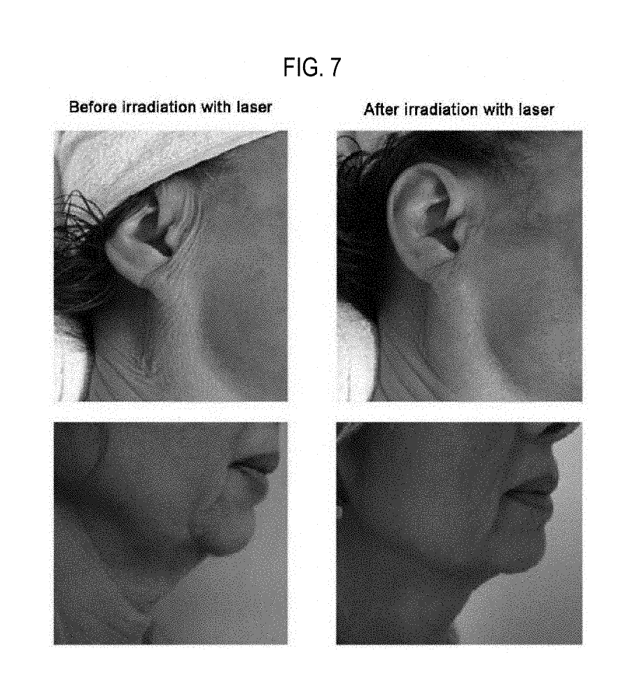

As for the results of the experiment, immediately after the laser irradiation, a clear reduction of wrinkles in the skin was visible and the elasticity of the skin was enhanced in the double chin and cheek areas, creating a prominent difference in the facial contour (FIG. 7).

### Reference Example 4: A Composition for Topical Fat Removal Comprising a Hyaluronidase and its Lipolytic Effect

In this study, a composition for topical fat removal comprising a hyaluronidase was administered to 40 (20 male and 20 female) volunteers selected from patients having abdominal obesity with a BMI of 25 or greater among adults of age 20 or above, in the form of injection given to their abdomen area. The effects of the composition for topical fat removal according to the present invention were determined as described below.

(1) Preparation of Injection Compositions for Topical Fat Removal Comprising Hyaluronidase

For the hyaluronidase, the Liporase Inj. of Daehan New Pharm Co., Ltd (1500 IU/vial, containing 13.3 mg lactose hydrate) was used, to which lidocaine (Hanmi Pharm. Co., Ltd.), Peniramin (Yuhan Co.), L-carnitine (Dream Pharma) and vitamin-C (Daewoo Pharm Co., Ltd.) were added in the amounts listed in the following table and mixed well to prepare 100 cc of a solution composition for injection.

TABLE 3
Ingredients of compositions for topical fat removal

| | Exp. | Exp. | | Exp. | | Exp. | | Comp. | | Comp. | | Comp. |
|---|---|---|---|---|---|---|---|---|---|---|---|---|
| Ingredients | Example 1 | Example 2 | | Example 3 | | Example 4 | | Example 1 | | Example 2 | | Example 3 |
| Hyaluronidase | 30,000 IU | 30,000 | IU | 60,000 | IU | 30,000 | IU | 25,000 | IU | 65,000 | IU | — |
| Lidocaine | — | 210 | mg | 210 | mg | 210 | mg | 210 | mg | 210 | mg | — |
| Peniramin | — | 12 | mg | 12 | mg | 12 | mg | 12 | mg | 12 | mg | — |

| | | | | | | | | | | | |
|---|---|---|---|---|---|---|---|---|---|---|---|
| L-Carnitine | — | — | 660 | mg | 660 | mg | 660 | mg | 660 | mg | — |

| | | | | | | | | | | |
|---|---|---|---|---|---|---|---|---|---|---|
| Vitamin C | — | — | — | 600 | mg | 600 | mg | 600 | mg | — |

| | | | | | | | |
|---|---|---|---|---|---|---|---|
| Saline solution | q.s. | q.s. | q.s. | q.s. | q.s. | q.s. | 100 cc |
| Total Amount | 100 | 100 | 100 | 100 | 100 | 100 | 100 |
| (cc) | | | | | | | |

(2) Lipolytic Effect of the Compositions for Topical Fat Removal

Changes in the waist circumference induced by the administration of the composition of Experimental Example 4 in Table 3 above, by the administration of the compositions of Comparative Examples 1 to 3, as well as by endomology, a conventional treatment for edema, were compared. In the abdominal area between the ribs and pubic bones, 1 cc each of a composition was injected at 50 points, arranged in a manner such that they are apart 1 cm each starting from the navel. The above injection was repeated weekly for four weeks, with the points of injection moved each time. Decreases in waist circumference were measured 1 week after completion of the above treatment, and the waist circumference was measured again 1 month after completion of the treatment. The average values of the measurements were recorded.

The endomology treatment, existing conventional treatment for edema, was carried out for four weeks, 30 sessions per week, with the decrease in waist circumference measured after one week and one month, and the results are shown in Table 4.

TABLE 4

| | Baseline (Day | 1 week after | | 1 month after | |
|---|---|---|---|---|---|
| (cm) | 0) | 4 sessions | Difference | 4 sessions | Difference |
| Comp. | 90.7 ± 1.2 | 91.0 ± 1.8 | +0.3 ± 1.2 | 90.5 ± 1.3 | −0.2 ± 1.2 |
| Example 3 | | | | | |
| Comp. | 91.0 ± 1.0 | 89.4 ± 0.4 | −1.6 ± 0.6 | 89.8 ± 1.3 | −1.2 ± 0.3 |
| Example 1 | | | | | |
| Comp. | 90.7 ± 1.3 | — | — | — | — |
| Example 2 | | | | | |
| Exp. | 90.8 ± 1.7 | 81.4 ± 0.5 | −9.4 ± 0.2 | 81.3 ± 0.3 | −9.5 ± 0.4 |
| Example 4 | | | | | |
| Endomology | 92.0 ± 2.0 | 89.8 ± 1.4 | −2.2 ± 0.8 | 92.2 ± 1.2 | +0.2 ± 1.5 |

For statistical analysis, the statistics package SPSS was used to obtain the distribution of measurements and the paired t-test was used to assess the effects of test injections on the decrease in waist circumference. The analysis used a 95% confidence interval, adjusted for weight changes.

In the above study, body fat analysis using the body fat analyzer Inbody showed an average fat loss of 1.5 to 2 kg.

According to Table 4, a change of −0.2±1.2 cm in waist circumference was observed in Comparative Example 3, where only a saline solution was given. In Experimental Example 4, however, a difference of −9.4±0.2 cm in waist circumference was seen at one week after the four sessions, and the difference was maintained as −9.5±0.4 cm at one month after the four sessions, indicating that there was no yo-yo effect.

On the other hand, in Comparative Example 1 where 250 IU of hyaluronidase was administered [a total of 12500 IU administered for 50 points per round of treatment; a total of 4 rounds of treatment], the difference was −1.6±0.6 cm at one week after the treatment and −1.2±0.3 cm at one month after the treatment, showing a significantly inferior effect than the present invention. In Comparative Example 2 where 650 IU of hyaluronidase was administered [a total of 32500 IU administered for 50 points per round of treatment; a total of 4 rounds of treatment], the treatment was discontinued after the first session due to dimpling and bruising.

In the case of endomology which is a treatment for edema, a decrease in waist circumference from the removal of edema was not significant and disappeared after one month, clearly indicating that a decrease in waist circumference obtained by using a hyaluronidase is not only a result of edema removal but also a result of lipolysis.

As can be seen from the above results, the injection composition of the present invention has been demonstrated to have an excellent effect in topical fat removal, with remarkably reduced side effects and no yo-yo effect.

In addition, the injection composition of the present invention has also been shown to have minimized side effects and an improved topical fat removal effect despite the use of a lower dose of hyaluronidase compared to the high dose hyaluronidase injection composition of the prior art KR 10-2009-0111916A.

(3) Assessment of Side Effects of the Administration of a Composition for Topical Fat Removal

The injection compositions of Experimental Examples 1 to 4 and those of Comparative Examples 1 to 3 were administered as in section (2) above, and the side effects observed in patients are listed in Table 5.

TABLE 5

| | | | | Pain above | | | | Skin |
|---|---|---|---|---|---|---|---|---|
| | Redness | Swelling | Itching | VAS 3 | Bruise | Dimpling | Sagging | elasticity |
| Comp. | 1 | 1 | 0 | 0 | 0 | 0 | 0 | 3 |
| Example 1 | | | | | | | | |
| Comp. | 4 | 3 | 4 | 2 | 10 | 15 | 0 | 2 |
| Example 2 | | | | | | | | |
| Comp. | 0 | 0 | 0 | 0 | 0 | 0 | 0 | 3 |
| Example 3 | | | | | | | | |
| Exp. | 11 | 10 | 11 | 13 | 1 | 0 | 1 | 3 |
| Example 1 | | | | | | | | |
| Exp. | 1 | 0 | 1 | 0 | 0 | 0 | 1 | 3 |
| Example 2 | | | | | | | | |
| Exp. | 1 | 1 | 0 | 0 | 0 | 1 | 1 | 3 |
| Example 3 | | | | | | | | |
| Exp. | 1 | 0 | 0 | 0 | 0 | 0 | 0 | 5 |
| Example 4 | | | | | | | | |

* Skin elasticity was determined by tactile assessment using a 5-point scale: 1: Very poor, 2: Poor, 3: Fair, 4: Good, 5: Very Good.

As shown in Table 5, with the composition of Experimental Example 1 according to the present invention, which does not contain local anesthetics or antihistamines, side effects such as bruise or dimpling were remarkably reduced compared to the high dose composition of Comparative Example 2, though pain and allergic reactions were observed. Moreover, the compositions of Experimental Examples 2, 3, 4 containing a local anesthetic and an antihistamine were shown to have remarkably reduced allergic redness, swelling, itching and pain compared to Experimental Example 1 or Comparative Example 2. These effects are thought to result from the use of a local anesthetic and an antihistamine as well as the low dose hyaluronidase. In addition, in the case of Experimental Example 4, excellent skin elasticity was observed, suggesting a potential skin elasticity improvement effect, which motivates combining with other anti-aging treatments.

Further, in Experimental Example 3, some patients developed slight dimpling and sagging as a result of fat removal by the lipolysis stimulator, which were limited to highly obese patients and recovered within a brief period.

In Experimental Example 4, the inclusion of a collagen production stimulator allowed the rapid recovery of skin resilience, with no occurrence of dimpling or sagging.

### Reference Example 5: A Skin Muscle Layer Irradiating Laser and its Elasticity Enhancing Effect

In this experiment, 9 volunteers among the participants who had received treatments to the step of Reference Example 3 underwent laser treatment. In this step, as the laser delivers energy to the muscle layer, it has excellent effects in collagen production, wrinkle improvement, and elasticity enhancement. Particularly, it can effectively deliver energy to the skin muscle layer by irradiating the laser beam from inside the mouth toward the face.

Specifically, a laser having a wavelength of 2940 nm and set at a frequency of 3 Hz (Fotona Laser-Slovenia) was used to irradiate the entire facial area from the oral mucosa toward the face for 5 minutes. This laser treatment is painless, and provides a thermal effect to the oral mucosa in a non-ablative, non-invasive manner without anesthesia to give a remodeling and tightening effect. By delivering well-controlled energy such that a temperature in the range of from 45 to 65° C. is reached in the upper dermis, the intraoral treatment of this step can lead to an instantaneous contraction of 30% of the tissue. The instantaneous contraction of the upper dermis mechanically pulls up the underlying deeper tissue layers, creating a lifting effect.

Since the above laser treatment delivers energy through the oral mucosa, it can protect the epidermis and stimulate collagen remodeling and neo-collagenesis (lasting up to 6 months). In addition, this laser treatment has the advantage that it is accompanied by no bleeding or pain except some warmth felt in the oral mucosa and there is no down time since oral mucosa is particularly quick to recover.

In the present experiment, the laser treatment was performed on 9 volunteers 5 times with an interval of one month. As a result, peri-oral wrinkles significantly decreased in all of the volunteers. Specifically, while the wrinkle score before treatment was 2.22 on the MFWS (Modified Fitzpatrick Wrinkle Scale), the score after treatment was 0.69 on the MFWS, showing an about 69% decrease in the wrinkle index.

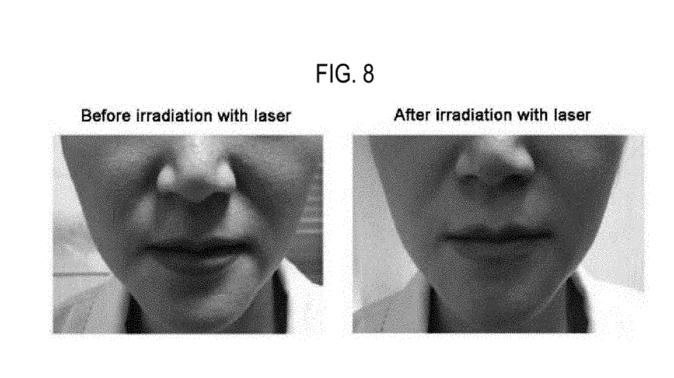

In addition, it was observed that the effect of the present step is shown instantaneously. With only one session of treatment using the frequency of 1.8 Hz, a significant reduction of the nasolabial wrinkles was visible (FIG. 8).

The present invention was conceived upon observing the effects of the treatments using A) a telomerase activating composition, B) a skin dermis irradiating laser, C) a composition for topical fat removal, and D) a skin muscle layer irradiating laser in the above Reference Examples. The present invention will now be described in detail with reference to the following examples. However, these examples are presented only to illustrate the present invention and should not be interpreted as limiting the present invention in any way.

### Examples

The present inventor completed the present invention by experimentally ascertaining that synergistic effects are exhibited when the treatments with A) a telomerase activating composition, B) a skin dermis irradiating laser, C) a composition for topical fat removal, and D) a skin muscle layer irradiating laser are combined, compared to the effects observed in the individual steps in the above Reference Examples. To comprehensively investigate the effects of the kit and aesthetic system for the prevention or treatment of skin aging according to the present invention, 90 subjects were treated with different combinations based on A) a telomerase activating composition, B) a skin dermis irradiating laser, C) a composition for topical fat removal, and D) a skin muscle layer irradiating laser, the components of the kit and an aesthetic system for the prevention of skin aging according to the present invention, and the collagen production/contraction/remodeling effect; elasticity improvement effect; wrinkle improvement effect; topical fat removal and facial contouring effect; telomerase activating effect; skin tone improvement effect; satisfaction and side effects were determined. Details of the study are described below.

(1) Subject Selection

The present study to investigate the effects of the kit and system for the prevention of skin aging was conducted in 90 male and female adult outpatients between 40 and 50 years of age who visited Dr. Sonyouna Clinic (Seoul, Korea) from October to November of 2016.

The subjects included in the present study were those who i) were informed of the purpose and study overview, procedures of the study and the requirements of the protocol, and signed an informed consent form. ii) were healthy, having no acute or chronic diseases including skin diseases, and iii) could be followed up during the study period.

The subjects were excluded if they fell under any one of the following groups:
1) pregnant or nursing women and women of childbearing potential,
2) people who have been using a topical preparation containing a steroid for the treatment of a skin disease,
3) people who have sensitive or hypersensitive skin,
4) people having skin legions such as moles, acne, erythema, telangiectasia, etc.
5) people who have used a functional cosmetics product for wrinkle improvement within 3 months before the start of the study,
6) people who have received a treatment on the study area within 6 months before the start of the study,
7) people who have or are on medication for an internal medical disease such as hypertension, diabetes, thyroid disease and who have cancer or other infectious diseases; a blood related disease; or an autoimmune disease.

(2) Methods

The subjects were randomized into groups and treated with different combinations of the components of the kit and aesthetic system of the present invention, (A), (B), (C), and (D).
(A) application of a telomerase activating composition: 3 cc of a telomerase activating composition containing 0.00056% (w/w) of a Kappaphycus alvarezii extract was applied, followed by irradiation with Legato (Alma Lasers, Israel), an ultrasound laser facilitating the deep penetration of cosmetics by ultrasound vibration, at a frequency of 30 Hz and 50% of maximum power output for 10 minutes over the entire face area in 1 to 2 passes.
(B) Irradiation with a skin dermis irradiating laser: A laser having a frequency of 5 Hz or lower (GentleMax) was irradiated for 3 minutes, and Indigo laser having a frequency of 30 to 50 Hz, was irradiated in mode A and mode B for 10 minutes respectively, followed by irradiation with the 92 w RF Tenor laser in the unipolar mode for 10 minutes.
(C) Injection of a composition for topical fat removal comprising a hyaluronidase: 100 cc of a composition for topical fat removal comprising 30,000 IU of hyaluronidase, 210 mg of lidocaine, 12 mg of Peniramin, 660 mg of L-carnitine, and 600 mg of vitamin-C, identical to Experimental Example 4 of Reference Example 4 described above, was prepared and a volume of 1 cc each, comprising 300 IU of hyaluronidase, was injected at 10 points with an interval between injection points of 1 cm.
(D) Irradiation of a skin muscle layer irradiating laser from inside the mouth toward the face: A Fotona laser, which irradiates the skin muscle layer at a frequency of 3 Hz was irradiated from inside the mouth toward the face for 6 minutes.

The following treatments were performed once on the respective subject groups, each consisting of 10 subjects.

TABLE 6
Treatments for subject groups

| | Treatments |
|---|---|
| Control 1 | A |
| Control 2 | B |
| Control 3 | C |
| Control 4 | D |
| Control 5 | A + B + C |
| Control 6 | A + B + D |
| Control 7 | A + B + C |
| Control 8 | B + C + D |
| Test group | A + B + C + D |

The collagen production/contraction/remodeling effect, skin elasticity enhancement effect, wrinkle improvement effect, facial contouring effect resulting from topical fat removal, topical fat removal/lipolysis in adipose cells/reduction of edema, telomerase activation in skin cells, and skin tone improvement effect were determined over time.

### Example 1: Determination of Collagen Production/Contraction/Remodeling Effect

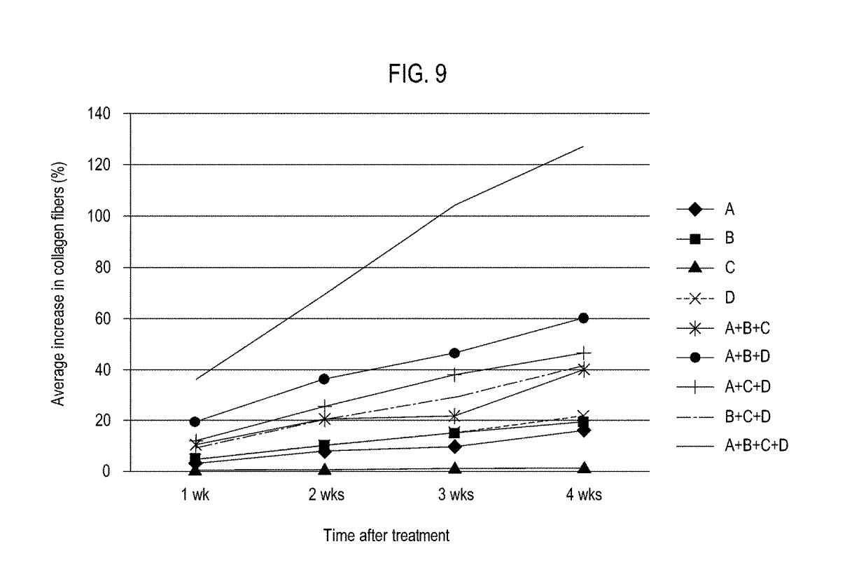

The skin biopsy specimens of the study subjects were collected to measure the number of collagen fibers. Specifically, skin biopsy specimens were fixed in 2.5% glutaraldehyde solution, washed with 0.1 M phosphate buffer (pH 7.4), and post-fixed in 1% osmium tetroxide solution for 2 hours. Next, the skin tissue was dehydrated with graded ethyl alcohol from 50% to anhydrous alcohol, infiltrated with propylene oxide, and then embedded in an Epon resin mixture. Thin survey sections with 1 μm thickness were cut to select areas of interest, and then ultrathin sections were obtained and treated with tannic acid to enhance the resolution of collagen fibers, followed by conventional double staining with uranyl acetate and lead citrate. The samples were then observed using a transmission electron microscope (JEOL 200CX, Japan), with all images taken at the same magnification (×40,000) to compare changes in the collagen fibers. The results are as summarized in Table 7 and illustrated in FIG. 9 using a graph.

TABLE 7
Increase in the average number of collagen fibers

| | Increase in average number of | | | |
|---|---|---|---|---|
| | collagen fibers in each group (%) | | | |
| | | 2 weeks | 3 weeks | 4 weeks |
| | 1 week post- | post- | post- | post- |
| | treatment | treatment | treatment | treatment |
| Control 1 - A | 3.3 | 7.9 | 9.9 | 16.1 |
| Control 2 - B | 4.6 | 10.1 | 15 | 19.3 |
| Control 3 - C | 0.7 | 0.95 | 1.1 | 1.4 |
| Control 4 - D | 4.1 | 10.2 | 15.7 | 21.7 |
| Control 5 - ABC | 10.3 | 20.4 | 21.5 | 39.7 |
| Control 6 - ABD | 19.6 | 36.0 | 46.4 | 60.0 |
| Control 7 - ACD | 12.2 | 25.3 | 38.1 | 46.3 |
| Control 8 - BCD | 9.3 | 20.2 | 29.3 | 41.4 |
| Test group - ABCD | 36.1 | 69.5 | 104.3 | 127.0 |

As demonstrated by Table 7 and FIG. 9, the test group according to the present invention (treated with the combination A, B, C, and D) showed a better collagen fiber increasing effect than all control groups from 1 week to 4 weeks after the treatment. Particularly, compared to the sum of the collagen fiber increasing effect in the single treatment groups, treated with A, B, C, or D (4 weeks post-treatment: increase in the number of fibers 58.5%), the test group showed a superior, synergistic effect (4 weeks post-treatment: 127.0%). Also, the test group showed a better effect than the sum of the effects found in the group treated with the combination ABC and a single treatment group treated with D (4 weeks post-treatment: 61.4%), the sum of the effects found in the group treated with the combination ABD and a single treatment group treated with C (4 weeks post-treatment: 61.4%), the sum of the effects found in the group treated with the combination ACD and a single treatment group treated with B (4 weeks post-treatment: 65.6%), and the sum of the effects found in the group treated with the combination BCD and a single treatment group treated with A (4 weeks post-treatment: 57.5%). Thus, by virtue of combining all of treatments A, B, C, and D, the aesthetic kit and aesthetic system of the present invention exhibited an excellent synergistic effect in increasing the number of collagen fibers.

### Example 2: Determination of the Elasticity Improvement Effect

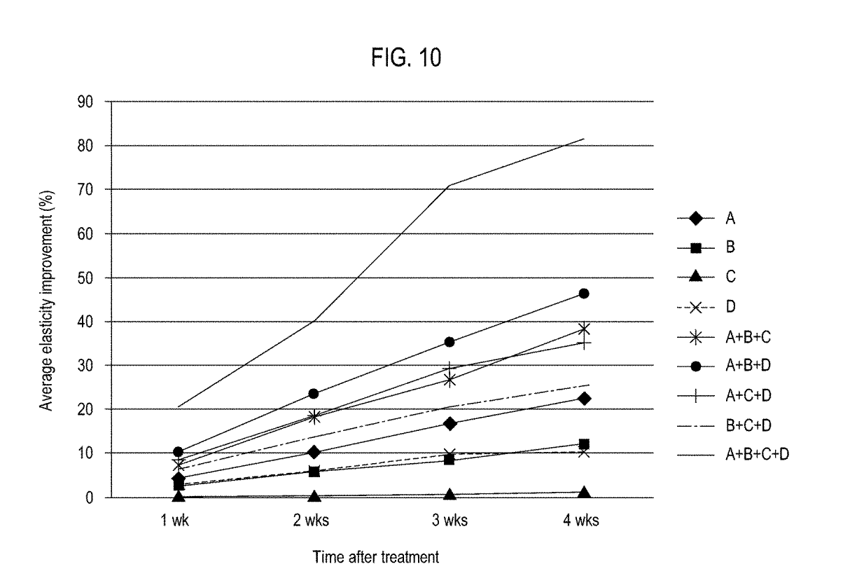

The skin elasticity of the study subjects was determined. Specifically, DermaLab USB elasticity probe (Cortex Technology, Inc.) was employed to evaluate the skin elasticity improvement in the study subjects. The DermaLab USB elasticity probe indicates the changes in the skin and the force of restoration due to the suction and duration of suction at the area of interest. The probe of the device was glued to the left cheek of test subjects using a special tape before taking the measurements. Considering that repeated measurements at the same site cause an elevated level of fatigue in the skin, each site of elasticity measurement was used only once. Changes in skin elasticity were analyzed using an application software (version 1.09) that is an exclusive analysis program for DermaLab USB. Among the measured parameters, Young's modulus (E) was used for the analysis, with a greater Young's modulus (E) indicating a higher elasticity. Young's modulus (E) is a value based on the deference of force needed when the skin surface is elevated 1.5 mm, the distance between two infrared detection lines in the measuring probe, and its unit of measurement is MegaPascal (MPa). The elasticity measurements were taken before treatment (baseline), as well as 1 week, 2 weeks, 3 weeks, and 4 weeks after treatment. The results are summarized in Table 8, and illustrated in FIG. 10 using a graph.

TABLE 8
Average skin elasticity improvement

| | Average elasticity improvement in each group (%) | | | |
|---|---|---|---|---|
| | 1 week | 2 weeks | | 4 weeks |
| | post- | post- | 3 weeks post- | post- |
| | treatment | treatment | treatment | treatment |
| Control 1 - A | 4.46 | 10.21 | 16.76 | 22.35 |
| Control 2 - B | 2.47 | 5.68 | 8.42 | 12.10 |
| Control 3 - C | 0.41 | 0.65 | 0.8 | 1.2 |
| Control 4 - D | 3.20 | 6.29 | 9.62 | 10.36 |
| Control 5 - ABC | 7.21 | 18.27 | 26.7 | 38.2 |
| Control 6 - ABD | 10.29 | 23.44 | 35.2 | 46.18 |
| Control 7 - ACD | 8.61 | 18.64 | 29.41 | 35.15 |
| Control 8 - BCD | 6.33 | 13.82 | 20.63 | 25.47 |
| Test group - ABCD | 20.72 | 40.25 | 70.89 | 81.50 |

As demonstrated by Table 8 and FIG. 10, the test group according to the present invention (treated with the combination A, B, C, and D) showed a better skin elasticity improvement effect than all control groups from 1 week to 4 weeks after the treatment. Particularly, compared to the sum of the skin elasticity improvement effects in the single treatment groups, treated with A, B, C, or D (4 weeks post-treatment: elasticity improvement 46.01%), the test group showed a superior effect (4 weeks post-treatment: 81.50%). Also, the test group showed a better effect than the sum of the effects found in the group treated with the combination ABC and a single treatment group treated with D (4 weeks post-treatment: 48.56%), the sum of the effects found in the group treated with the combination ABD and a single treatment group treated with C (4 weeks post-treatment: 36.35%), the sum of the effects found in the group treated with the combination ACD and a single treatment group treated with B (4 weeks post-treatment: 47.25%), and the sum of the effects found in the group treated with the combination BCD and a single treatment group treated with A (4 weeks post-treatment: 47.82%). Thus, it has been demonstrated that by virtue of the organic combination of treatments A, B, C, and D, the kit and aesthetic system of the present invention for the prevention of skin aging exhibit an excellent synergistic effect in skin elasticity improvement.

### Example 3: Assessment of the Wrinkle Improvement Effect

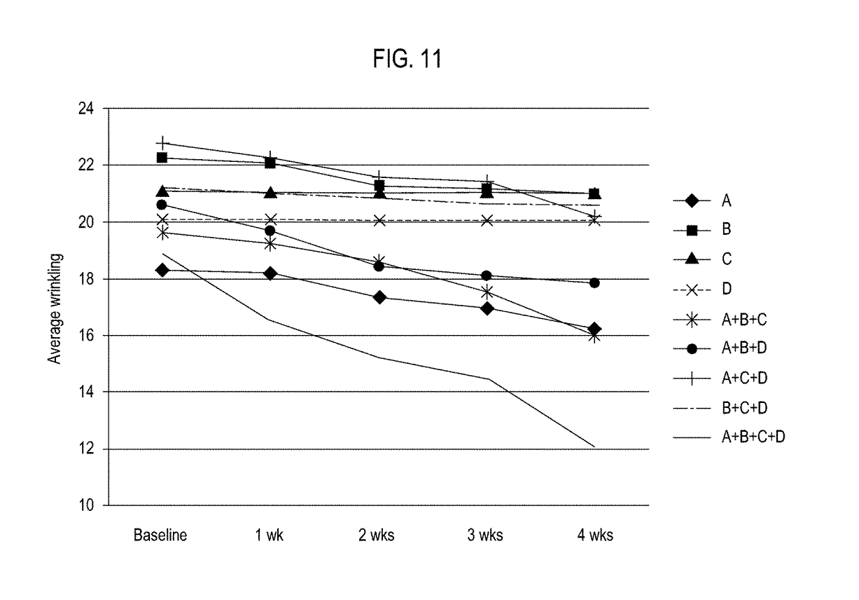

The wrinkle improvement effect in the study subjects were determined. For a quantitative assessment of wrinkling, a device quantifying the extent of wrinkling (Aramo TS, programmed by Aram HUVIS Co., South Korea) was used. The head of a Beauty scope (×10 lens) with a diameter of 30 mm was placed in close contact with the skin surface of the area of interest, and pressed perpendicularly onto the skin with constant pressure. Then, differences in the brightness of the skin generated by the light from the device head were used to quantify the wrinkling. A negative model of skin wrinkles thus obtained can be transformed into a positive format using a software, or parameter values can be adjusted for diverse measurement lines. As for the extent of wrinkling, a value of 10˜12 indicates normal wrinkling for people in their twenties. The results are summarized in Table 9, and illustrated in FIG. 11 using a graph.

TABLE 9
Average wrinkling

| | Average wrinkling in each group | | | | |
|---|---|---|---|---|---|
| | | 1 week post- | 2 weeks post- | 3 weeks post- | 4 weeks post- |
| | Baseline | treatment | treatment | treatment | treatment |
| Control 1 - A | 18.30 | 18.20 | 17.34 | 16.95 | 16.20 |
| Control 2 - B | 22.25 | 22.10 | 21.26 | 21.11 | 20.95 |
| Control 3 - C | 21.14 | 21.04 | 21.00 | 21.04 | 21.00 |
| Control 4 - D | 20.10 | 20.08 | 20.06 | 20.05 | 20.04 |
| Control 5 - ABC | 19.63 | 19.24 | 18.61 | 17.54 | 16.02 |
| Control 6 - ABD | 20.60 | 19.68 | 18.45 | 18.11 | 17.80 |
| Control 7 - ACD | 22.81 | 22.23 | 21.59 | 21.44 | 20.21 |
| Control 8 - BCD | 21.22 | 21.00 | 20.85 | 20.64 | 20.58 |
| Test group - ABCD | 18.89 | 16.52 | 15.23 | 14.47 | 12.08 |

As demonstrated by Table 9 and FIG. 11, the test group according to the present invention (treated with the combination A, B, C, and D) showed a better wrinkle improvement effect than all control groups from 1 week to 4 weeks after the treatment. Particularly, compared to the sum of the wrinkle improvement effects in the single treatment groups, treated with A, B, C, or D (4 weeks post-treatment: wrinkle reduction −3.60), the test group showed a superior, synergistic effect (4 weeks post-treatment: −6.81). Also, the test group showed a better effect than the sum of the effects found in the group treated with the combination ABC and a single treatment group treated with D (4 weeks post-treatment: −3.67), the sum of the effects found in the group treated with the combination ABD and a single treatment group treated with C (4 weeks post-treatment: −2.94), the sum of the effects found in the group treated with the combination ACD and a single treatment group treated with B (4 weeks post-treatment: −3.90), and the sum of the effects found in the group treated with the combination BCD and a single treatment group treated with A (4 weeks post-treatment: −2.74). Thus, by virtue of combining all of treatments A, B, C, and D, the kit and aesthetic system of the present invention for the prevention of skin aging have an excellent synergistic effect in wrinkle improvement.

### Example 4: Assessment of the Skin Contouring Effect Resulting from Topical Fat Removal and Reduction of Edema

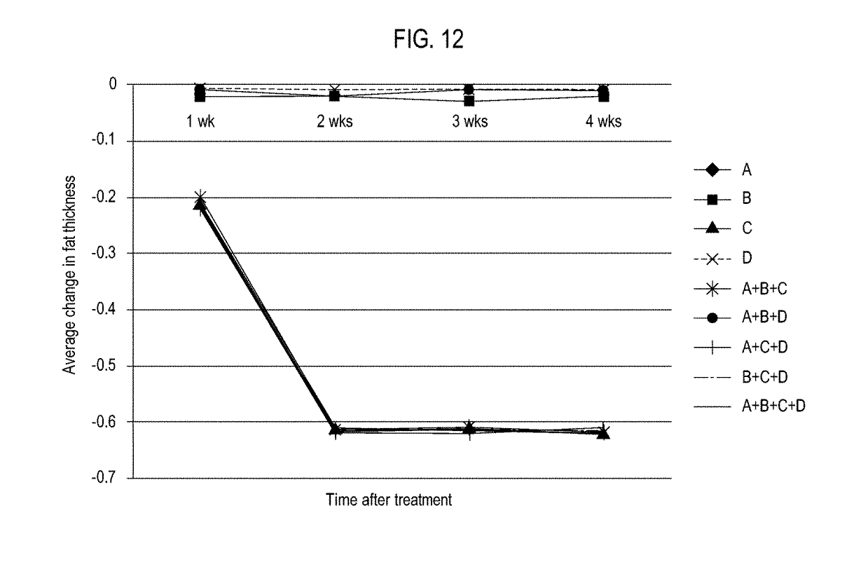

The skin contouring effect resulting from the administration of a composition for topical fat removal etc., was determined in the study subjects. Specifically, ultrasound was used to measure the thickness of subcutaneous fat in the face area of study subjects. A SuperSonic Imagine (Les Jardins de la Duranne, Aix en Provence, France) device was used for the ultrasound based measurement. The tester and the subject sat facing each other to measure the thickness of subcutaneous fat in the cheek and chin areas, and the pressure on the ultrasound device was minimized to correctly measure the thickness. An aqueous gel was applied to the site of measurement to help transmit the ultrasound, and a high frequency linear probe at 6.0-MHz was used to take measurements at the two sites, followed by calculation of the average value. The results are summarized in Table 10, and illustrated in FIG. 12 using a graph.

TABLE 10
Changes in the thickness of fat in the chin area

| | Average changes in the fat | | | |
|---|---|---|---|---|
| | thickness in each group (cm) | | | |
| | 1 week | 2 weeks | | 4 weeks |
| | post- | post- | 3 weeks post- | post- |
| | treatment | treatment | treatment | treatment |
| Control 1 - A | −0.01 | −0.02 | −0.01 | −0.01 |
| Control 2 - B | −0.02 | −0.02 | −0.03 | −0.02 |
| Control 3 - C | −0.21 | −0.61 | −0.61 | −0.62 |
| Control 4 - D | −0.01 | −0.01 | −0.01 | −0.01 |
| Control 5 - ABC | −0.2 | −0.61 | −0.61 | −0.62 |
| Control 6 - ABD | −0.01 | −0.02 | −0.01 | −0.01 |
| Control 7 - ACD | −0.22 | −0.62 | −0.62 | −0.61 |
| Control 8 - BCD | −0.21 | −0.61 | −0.61 | −0.62 |
| Test group - ABCD | −0.21 | −0.61 | −0.62 | −0.62 |

As demonstrated by Table 10 and FIG. 12, the test group according to the present invention (treated with the combination A, B, C, and D) showed an excellent fat thickness reducing effect from 1 week to 4 weeks after the treatment. It can be seen that this effect depends on the inclusion of the treatment with C (a composition for topical fat removal), an element of the present invention, and a prominent synergistic effect can be observed from 1 week after treatment, which lasts until 4 weeks after treatment.

### Example 5: Determination of the Telomerase Activating Effect

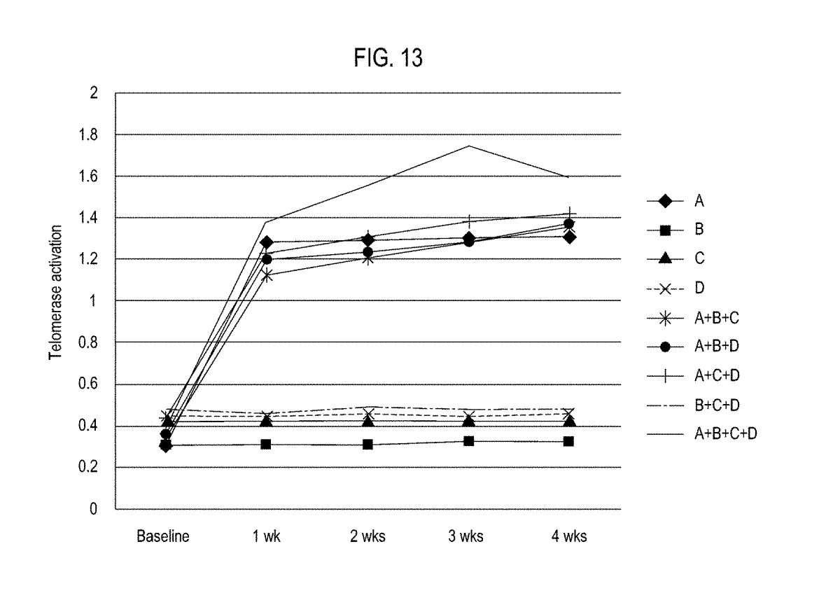

The extent of telomerase activation was assessed in the study subjects. The study subjects abstained from caffeine, excessive medication or metabolism regulators for 24 hours before their blood test, and blood samples were drawn at 10 AM after they took enough rest. A sandwich enzyme immunoassay was used to measure the amount of telomerase in the samples. Specifically, a standard or test specimen containing telomerase was applied to a plate coated with a monoclonal antibody (MAb) specific to telomerase (TA), thereby attaching telomerase to the plate. To quantify the amount of telomerase in the specimen, an HRP (horseradish peroxidase) conjugated polyclonal antibody that specifically binds to telomerase is prepared and applied to the plate, resulting in its binding to the telomerase associated with the plate, in a sandwich-like format. When all the reactions were completed upon incubation, the excess substances remaining unreacted on the plate were washed off, and a substrate solution was reacted to determine the amount of bound HRP. When the substrate is reacted with HRP, it is hydrolyzed by the peroxidase activity of HRP, resulting in the development of color in the substrate solution. This reaction between the enzyme and substrate was terminated by the addition of sulphuric acid, and the colored substrate solution was measured with a spectrophotometer at a wavelength of 450 nm to create a standard curve to calculate the concentration of telomerase. The results are summarized in Table 11, and illustrated in FIG. 13 using a graph.

TABLE 11
Telomerase activity improvement

| | Average telomerase activity in each group | | | | |
|---|---|---|---|---|---|
| | | | | 3 weeks | 4 weeks |
| | | 1 week post- | 2 weeks post- | post- | post- |
| | Baseline | treatment | treatment | treatment | treatment |
| Control 1 - A | 0.30 ± 0.23 | 1.28 ± 0.23 | 1.29 ± 0.14 | 1.30 ± 0.78 | 1.31 ± 0.46 |
| Control 2 - B | 0.31 ± 0.58 | 0.31 ± 0.25 | 0.31 ± 0.11 | 0.32 ± 0.64 | 0.32 ± 0.21 |
| Control 3 - C | 0.42 ± 0.24 | 0.42 ± 0.49 | 0.42 ± 0.46 | 0.42 ± 0.24 | 0.42 ± 0.11 |
| Control 4 - D | 0.45 ± 0.16 | 0.45 ± 0.65 | 0.46 ± 0.77 | 0.45 ± 0.31 | 0.46 ± 0.14 |
| Control 5 - ABC | 0.35 ± 0.68 | 1.13 ± 0.27 | 1.21 ± 0.54 | 1.29 ± 0.29 | 1.36 ± 0.41 |
| Control 6 - ABD | 0.36 ± 0.42 | 1.20 ± 0.81 | 1.23 ± 0.16 | 1.28 ± 0.64 | 1.37 ± 0.32 |
| Control 7 - ACD | 0.44 ± 0.28 | 1.23 ± 0.46 | 1.31 ± 0.94 | 1.38 ± 0.55 | 1.42 ± 0.27 |
| Control 8 - BCD | 0.48 ± 0.22 | 0.46 ± 0.27 | 0.49 ± 0.51 | 0.48 ± 0.27 | 0.48 ± 0.42 |
| Test group - ABCD | 0.39 ± 0.67 | 1.38 ± 0.91 | 1.56 ± 0.67 | 1.75 ± 0.29 | 1.59 ± 0.68 |

As demonstrated by Table 11 and FIG. 13, the test group according to the present invention (treated with the combination A, B, C, and D) showed a better telomerase activating effect than all control groups from 1 week to 4 weeks after the treatment. Particularly, compared to the sum of the telomerase activating effects in the single treatment groups, treated with A, B, C, or D (4 weeks post-treatment: telomerase activity increase 1.03), the test group showed a superior, synergistic effect (4 weeks post-treatment: 1.20). Also, the test group showed a better, synergistic effect than the sum of the effects found in the group treated with the combination ABC and a single treatment group treated with D (4 weeks post-treatment: 1.02), the sum of the effects found in the group treated with the combination ABD and a single treatment group treated with C (4 weeks post-treatment: 1.01), the sum of the effects found in the group treated with the combination ACD and a single treatment group treated with B (4 weeks post-treatment: 0.99), and the sum of the effects found in the group treated with the combination BCD and a single treatment group treated with A (4 weeks post-treatment: 1.01). Thus, by virtue of combining all of treatments A, B, C, and D, the kit and aesthetic system of the present invention for the prevention of skin aging have an excellent effect on telomerase activation.

### Example 6: Determination of the Skin Tone Improvement Effect

(1) Measurement of Skin Hydration

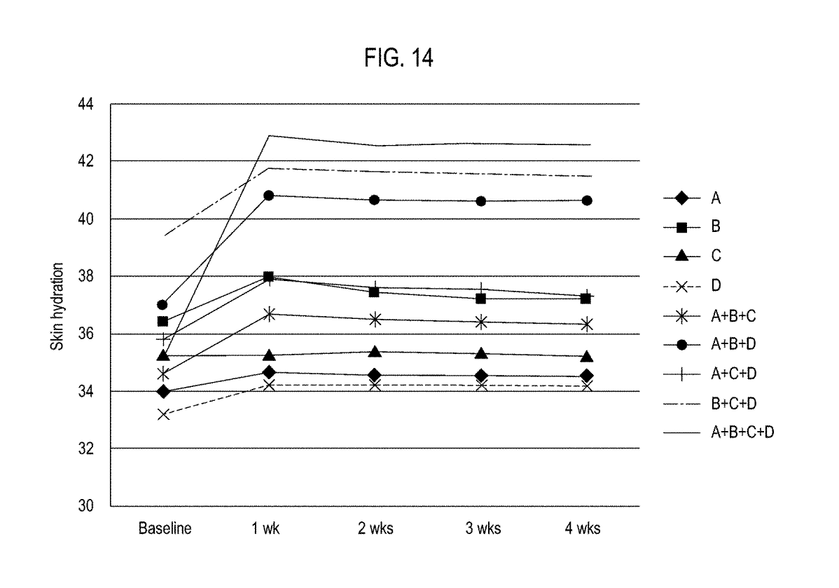

The skin hydration of the study subjects was determined. This is done by measuring the capacitance reflecting the content of water, which has the highest dielectric constant in the skin, indicated by an arbitrary unit (AU). That is, skin hydration is usually determined by measuring the capacitance of the weak current transmitted to a probe contacting the skin surface. As the water content and capacitance are proportional to each other, the greater the skin hydration, the higher the measurement values are. This measuring device thus can consistently measure the water content within the depth of 30˜40 μm underneath the stratum corneum under a condition that is not substantially affected by the application of cosmetics or medications (conductor track in electric scatter field). In addition, the device has a unique capacity to assess skin hydration under special test conditions, e.g., when the probe is maintaining a slight distance from the skin without directly contacting it. The readings of the device range from a minimum of 0 AU to 120 AU. In this regard, the capacitance of a solid is generally 7 F (farad), and the capacitance of water is 81 F. Then, if a weak current between 7 F and 81 F flows, differences in the capacitance will appear. This difference is divided by 120 to give the values of the above arbitrary unit (AU). According to this principle, plastics which generally do not contain any water are shown to have a capacitance of about 6˜8 AU when measured by the device, and 100% moisture would give 120 AU when measured. That is, a higher value is interpreted to indicate a higher water content. A numerical reading appears when a probe with a diameter of 16 mm was placed at the site of measurement in close contact with the skin surface and pressed perpendicularly onto the skin with a constant pressure, and the average of 5 measurements taken within a certain area (central region between the cheek bone and chin, 4×4 cm) was used for the record. A reading of 35˜40 indicates a good degree of hydration, 31˜34 indicates a slight lack of moisture, and 25˜30 indicates a moisture-lacking condition. The results are summarized in Table 12, and illustrated in FIG. 14 using a graph.

TABLE 12
Skin hydration improvement

| | Average skin hydration in each group | | | | |
|---|---|---|---|---|---|
| | | 1 week post- | 2 weeks post- | 3 weeks post- | 4 weeks post- |
| | Baseline | treatment | treatment | treatment | treatment |
| Control 1 - A | 34.00 | 34.67 | 34.54 | 34.55 | 34.52 |
| Control 2 - B | 36.41 | 37.98 | 37.42 | 37.20 | 37.22 |
| Control 3 - C | 35.26 | 35.24 | 35.38 | 35.30 | 35.22 |
| Control 4 - D | 33.21 | 34.22 | 34.20 | 34.22 | 34.19 |
| Control 5 - ABC | 34.60 | 36.68 | 36.52 | 36.40 | 36.33 |
| Control 6 - ABD | 37.00 | 40.83 | 40.63 | 40.61 | 40.60 |
| Control 7 - ACD | 35.80 | 37.87 | 37.60 | 37.54 | 37.31 |
| Control 8 - BCD | 39.41 | 41.76 | 41.66 | 41.57 | 41.50 |
| Test group - ABCD | 35.21 | 42.89 | 42.55 | 42.64 | 42.60 |

As demonstrated by Table 12 and FIG. 14, the test group according to the present invention (treated with the combination A, B, C, and D) showed a better skin hydration improvement effect than all control groups from 1 week to 4 weeks after the treatment. Particularly, compared to the sum of the skin hydration improvement effects in the single treatment groups, treated with A, B, C, or D (4 weeks post-treatment: increase in skin hydration 2.27), the test group showed a superior, synergistic effect (4 weeks post-treatment: 7.39). Also, the test group showed a better, synergistic effect than the sum of the effects found in the group treated with the combination ABC and a single treatment group treated with D (4 weeks post-treatment: 2.71), the sum of the effects found in the group treated with the combination ABD and a single treatment group treated with C (4 weeks post-treatment: 3.56), the sum of the effects found in the group treated with the combination ACD and a single treatment group treated with B (4 weeks post-treatment: 2.32), and the sum of the effects found in the group treated with the combination BCD and a single treatment group treated with A (4 weeks post-treatment: 2.61). Thus, by virtue of combining all of treatments A, B, C, and D, the kit and aesthetic system of the present invention for the prevention of skin aging have an excellent effect in skin hydration improvement.

(2) Measurement of Skin Roughness

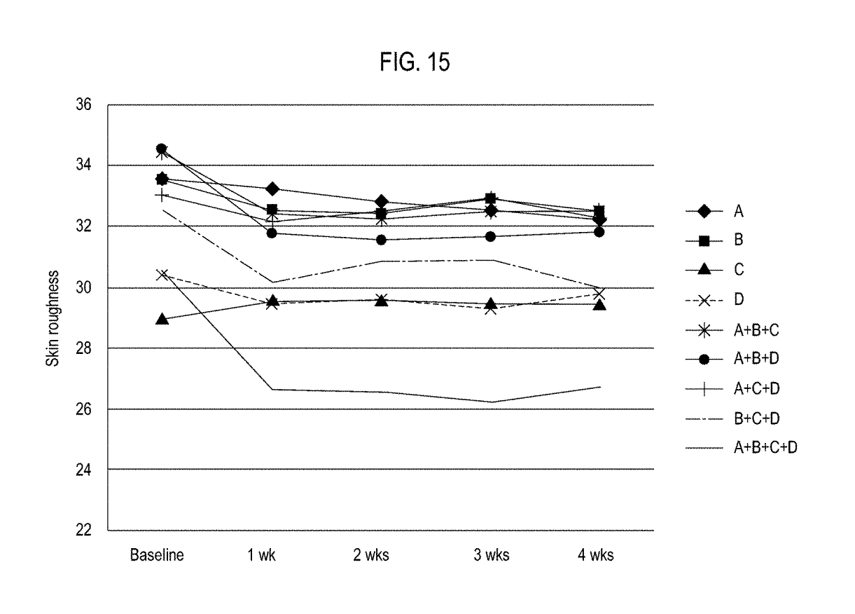

To assess the skin roughness of the study subjects, images of the test areas were taken using optical principles, and the skin surface contour was transformed into a graph and numbers. A Beauty scope (×60 lens) with a diameter of 30 mm was used for this, and the head of the device was placed at the site of measurement in close contact with the skin surface and pressed perpendicularly onto the skin with constant pressure, and the average of 5 measurements taken within a certain area (central region between the cheek bone and chin, 4×4 cm) was used for the record. A reading of 28˜31 indicates that the roughness is good, 32˜35 indicates that the skin is slightly rough, and 36˜40 indicates that the skin is rough. The results are summarized in Table 13, and illustrated in FIG. 15 using a graph.

TABLE 13
Skin roughness

| | Average skin roughness in each group | | | | |
|---|---|---|---|---|---|
| | | 1 week post- | 2 weeks post- | 3 weeks post- | 4 weeks post- |
| | Baseline | treatment | treatment | treatment | treatment |
| Control 1 - A | 33.59 | 33.25 | 32.82 | 32.56 | 32.21 |
| Control 2 - B | 33.50 | 32.56 | 32.41 | 32.89 | 32.51 |
| Control 3 - C | 29.00 | 29.56 | 29.56 | 29.47 | 29.44 |
| Control 4 - D | 30.40 | 29.47 | 29.58 | 29.34 | 29.78 |
| Control 5 - ABC | 34.40 | 32.40 | 32.25 | 32.47 | 32.47 |
| Control 6 - ABD | 34.50 | 31.78 | 31.56 | 31.64 | 31.82 |
| Control 7 - ACD | 33.05 | 32.14 | 32.52 | 32.89 | 32.29 |
| Control 8 - BCD | 32.56 | 30.16 | 30.84 | 30.89 | 30.00 |
| Test group - ABCD | 30.56 | 26.67 | 26.56 | 26.23 | 26.74 |

As demonstrated by Table 13 and FIG. 15, the test group according to the present invention (treated with the combination A, B, C, and D) showed a better skin roughness reducing effect than all control groups from 1 week to 4 weeks after the treatment. Particularly, compared to the sum of the skin roughness reducing effects in the single treatment groups, treated with A, B, C, or D (4 weeks post-treatment: skin roughness reduction −2.17), the test group showed a superior, synergistic effect (4 weeks post-treatment: −3.82). Also, the test group showed a better, synergistic effect than the sum of the effects found in the group treated with the combination ABC and a single treatment group treated with D (4 weeks post-treatment: −2.55), the sum of the effects found in the group treated with the combination ABD and a single treatment group treated with C (4 weeks post-treatment: −2.21), the sum of the effects found in the group treated with the combination ACD and a single treatment group treated with B (4 weeks post-treatment: −1.75), and the sum of the effects found in the group treated with the combination BCD and a single treatment group treated with A (4 weeks post-treatment: −3.56). Thus, by virtue of combining all of treatments A, B, C, and D, the kit and aesthetic system of the present invention for the prevention of skin aging have an excellent, synergistic effect in skin roughness reduction.

(3) Measurement of Pigmentation and Pores

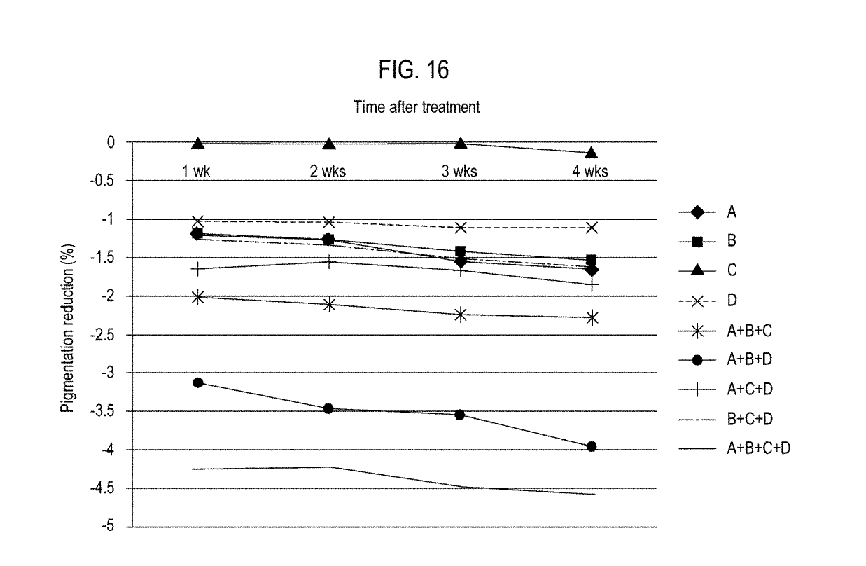

A RSA (Robo Skin Analyzer CS 50, In Forward. Inc., Japan) was used to measure changes in the amount of pigmentation in the study subjects. RSA is an instrument that allows for a combined examination of pores, pigmentation, wrinkles, skin tone, etc. It can take photographs from an identical angle and lighting by using the Compact booth, and has a high reproducibility. The pores and changes in the amount of pigmentation in the study subjects were measured with this instrument, which, consisting of two parts, an image capturing device and an image analyzing computer system, captures the image information on wrinkles, pigment, skine tone, pores, etc. and directly produces the results. It enables an accurate analysis as it can take photographs from three different angles. The results are summarized in Table 14, and illustrated in FIG. 16 using a graph.

TABLE 14
Pigmentation reduction (%)

| | Average pigmentation reduction in each group (%) | | | |
|---|---|---|---|---|
| | 1 week | 2 weeks | | 4 weeks |
| | post- | post- | 3 weeks post- | post- |
| | treatment | treatment | treatment | treatment |
| Control 1 - A | −1.22 | −1.26 | −1.54 | −1.64 |
| Control 2 - B | −1.19 | −1.28 | −1.42 | −1.54 |
| Control 3 - C | −0.01 | −0.02 | −0.01 | −0.13 |
| Control 4 - D | −1.02 | −1.04 | −1.10 | −1.11 |
| Control 5 - ABC | −2.00 | −2.11 | −2.24 | −2.28 |
| Control 6 - ABD | −3.13 | −3.47 | −3.55 | −3.96 |
| Control 7 - ACD | −1.65 | −1.56 | −1.66 | −1.84 |
| Control 8 - BCD | −1.26 | −1.33 | −1.52 | −1.62 |
| Test group - ABCD | −4.25 | −4.22 | −4.48 | −4.58 |

As demonstrated by Table 14 and FIG. 16, the test group according to the present invention (treated with the combination A, B, C, and D) showed a better pigmentation reducing effect than all control groups from 1 week to 4 weeks after the treatment. The test group showed a prominently superior, synergistic effect (4 weeks post-treatment: −4.58) than the sum of the effects found in the group treated with the combination ABC and a single treatment group treated with D (4 weeks post-treatment: −3.39), the sum of the effects found in the group treated with the combination ABD and a single treatment group treated with C (4 weeks post-treatment: −3.83), the sum of the effects found in the group treated with the combination ACD and a single treatment group treated with B (4 weeks post-treatment: −3.38), and the sum of the effects found in the group treated with the combination BCD and a single treatment group treated with A (4 weeks post-treatment: −3.26). Thus, by virtue of combining all of treatments A, B, C, and D, the kit and aesthetic system of the present invention for the prevention of skin aging have a significantly excellent, synergistic effect in skin pigmentation reduction.

### Example 7: Evaluation Questionnaire for Post-Treatment Changes in the Skin and Satisfaction, and Side Effects

An evaluation questionnaire survey was conducted with respect to the post-treatment changes in the skin and satisfaction of the study subjects at 4 weeks after treatment. The questionnaire consisted of 8 questions, including 7 questions on the evaluation of satisfaction on the changes in the skin and 1 question on the intention to use in the future, to the survey subjects' self-assessment after treatment. In addition, the study areas were observed for adverse skin reactions such as erythema, edema, scaling, itching, and prickling, and the results are summarized in Table 15.

TABLE 15

| | | | Frequency | | | | | | | | | |
|---|---|---|---|---|---|---|---|---|---|---|---|---|
| | | | A | B | C | D | ABC | ABD | ACD | BCD | ABCD | |
| Elasticity | Very unsatisfied | | 1 | 1 | 6 | 1 | 0 | 0 | 1 | 1 | 0 | 11 |
| improvement | Unsatisfied | | 4 | 3 | 4 | 4 | 4 | 1 | 3 | 4 | 0 | 27 |
| effect | Neutral | | 3 | 5 | 0 | 4 | 3 | 4 | 3 | 1 | 1 | 24 |
| | Satisfied | | 2 | 1 | 0 | 1 | 2 | 3 | 2 | 3 | 3 | 17 |
| | Very satisfied | | 0 | 0 | 0 | 0 | 1 | 2 | 1 | 1 | 6 | 11 |
| Wrinkle | Very unsatisfied | | 0 | 0 | 3 | 2 | 0 | 0 | 3 | 2 | 0 | 10 |
| improvement | Unsatisfied | | 4 | 3 | 5 | 6 | 2 | 2 | 5 | 5 | 0 | 32 |
| effect | Neutral | | 3 | 4 | 2 | 2 | 3 | 3 | 1 | 3 | 1 | 22 |
| | Satisfied | | 2 | 2 | 0 | 0 | 3 | 2 | 1 | 0 | 6 | 16 |
| | Very satisfied | | 1 | 1 | 0 | 0 | 2 | 3 | 0 | 0 | 3 | 10 |
| Contouring | Very unsatisfied | | 5 | 4 | 0 | 5 | 0 | 4 | 0 | 0 | 0 | 18 |
| effect | Unsatisfied | | 4 | 3 | 0 | 4 | 0 | 5 | 0 | 0 | 0 | 16 |
| | Neutral | | 1 | 3 | 1 | 1 | 1 | 1 | 0 | 1 | 0 | 9 |
| | Satisfied | | 0 | 0 | 3 | 0 | 3 | 0 | 3 | 2 | 3 | 14 |
| | Very satisfied | | 0 | 0 | 6 | 0 | 6 | 0 | 7 | 7 | 7 | 33 |
| Hydration | Very unsatisfied | | 2 | 1 | 6 | 1 | 0 | 0 | 0 | 0 | 0 | 10 |
| improvement | Unsatisfied | | 3 | 3 | 3 | 4 | 2 | 0 | 2 | 4 | 0 | 21 |
| effect | Neutral | | 3 | 4 | 1 | 3 | 4 | 5 | 6 | 5 | 1 | 32 |
| | Satisfied | | 2 | 2 | 0 | 2 | 3 | 4 | 1 | 0 | 5 | 19 |
| | Very satisfied | | 0 | 0 | 0 | 0 | 1 | 1 | 1 | 1 | 4 | 8 |
| Roughness | Very unsatisfied | | 0 | 0 | 7 | 0 | 1 | 0 | 0 | 0 | 0 | 8 |
| improvement | Unsatisfied | | 2 | 3 | 2 | 3 | 2 | 1 | 3 | 3 | 0 | 19 |
| effect | Neutral | | 4 | 5 | 1 | 3 | 2 | 3 | 1 | 1 | 1 | 21 |
| | Satisfied | | 1 | 0 | 0 | 1 | 3 | 4 | 2 | 3 | 1 | 15 |
| | Very satisfied | | 3 | 2 | 0 | 3 | 2 | 2 | 4 | 3 | 8 | 27 |
| Pigmentation | Very unsatisfied | | 4 | 2 | 9 | 3 | 2 | 0 | 2 | 1 | 0 | 23 |
| improvement | Unsatisfied | | 1 | 4 | 1 | 2 | 4 | 2 | 3 | 5 | 1 | 24 |
| effect | Neutral | | 5 | 4 | 0 | 5 | 3 | 4 | 4 | 3 | 5 | 32 |
| | Satisfied | | 0 | 0 | 0 | 0 | 1 | 3 | 1 | 1 | 3 | 9 |
| | Very satisfied | | 0 | 0 | 0 | 0 | 0 | 1 | 0 | 0 | 1 | 2 |
| Satisfaction | Very unsatisfied | | 1 | 0 | 0 | 1 | 0 | 0 | 0 | 0 | 0 | 2 |
| about T-CAT | Unsatisfied | | 4 | 2 | 2 | 5 | 3 | 2 | 5 | 3 | 0 | 26 |
| treatment | Neutral | | 5 | 6 | 5 | 3 | 3 | 4 | 2 | 2 | 1 | 31 |
| | Satisfied | | 0 | 2 | 1 | 1 | 2 | 1 | 1 | 3 | 4 | 15 |
| | Very satisfied | | 0 | 0 | 2 | 0 | 2 | 3 | 2 | 2 | 5 | 16 |
| Intention to | Strong no | | 1 | 0 | 0 | 1 | 0 | 0 | 0 | 0 | 0 | 2 |
| use in the | No | | 3 | 4 | 1 | 5 | 1 | 1 | 1 | 2 | 0 | 18 |
| future | Neutral | | 5 | 4 | 4 | 3 | 5 | 5 | 3 | 4 | 0 | 33 |
| | Yes | | 1 | 2 | 2 | 1 | 2 | 3 | 4 | 2 | 2 | 19 |
| | Strong yes | | 0 | 0 | 3 | 0 | 2 | 1 | 2 | 2 | 8 | 18 |
| Adverse skin | Yes | Erythema | 0 | 0 | 1 | 0 | 0 | 0 | 1 | 1 | 1 | 4 |
| reactions | | Edema | 0 | 0 | 0 | 0 | 0 | 0 | 0 | 0 | 0 | 0 |
| experienced | | Scaling | 0 | 0 | 0 | 0 | 0 | 0 | 0 | 0 | 0 | 0 |
| after | | Itching | 0 | 0 | 1 | 0 | 1 | 0 | 1 | 1 | 1 | 5 |
| treatment | | Burning | 0 | 0 | 0 | 0 | 0 | 0 | 0 | 0 | 0 | 0 |
| | | sensation | | | | | | | | | | |

| | | | | | | | | | |
|---|---|---|---|---|---|---|---|---|---|
| No | 90 | 90 | 88 | 90 | 89 | 90 | 88 | 88 | 88 |

As can be seen in Table 15, the kit or aesthetic system for the prevention of skin aging according to the present invention (test group—treatment with ABCD) got the highest satisfaction for all self-assessment of effect items.

These self-assessment of effect results are consistent with the results of collagen fiber increase, elasticity improvement, wrinkle improvement, skin contouring effect, hydration improvement, roughness improvement, and pigmentation improvement which were numerically shown in Examples 1 to 6, demonstrating that the kit or aesthetic system for the prevention of skin aging according to the present invention has synergistic effects in various aspects related to skin aging, by virtue of comprising the components A, B, C, and D. Accordingly, 80% of the subjects in the group treated with the combination ABCD, the kit for the prevention of skin aging according to the present invention, indicated a strong intention to use in the future, while less than 30% of the subjects in the control groups indicated an intention to use in the future.

Meanwhile, one subject each from the C single treatment group, ABD combination treatment group, ACD combination treatment group, and BCD combination treatment group experienced erythema, and one subject each from the C single treatment group, D single treatment group, ABD combination treatment group, ACD combination treatment group, and BCD combination treatment group experienced itching. However, no significant difference between the treatment groups was noticeable.

A person skilled in the art to which the present invention pertains would understand from the foregoing descriptions that the present invention can be practiced in other embodiments without departing from its technical idea or essential features. In this connection, the foregoing examples should be interpreted to be illustrative only and non-limiting in any respect. Thus, the scope of protection for the present invention will be determined by the accompanying claims and equivalents thereof

What is claimed is:

1. A kit for the prevention of skin aging, comprising
A) a telomerase activating composition;
B) a skin dermis irradiating laser;
C) a composition for topical fat removal comprising 300 IU to 600 IU of hyaluronidase; and
D) a skin muscle layer irradiating laser.

2. The kit of claim 1, wherein the prevention of skin aging is one or more of collagen production, collagen contraction, collagen remodeling, skin contouring, skin elasticity enhancement, wrinkle improvement, topical fat removal, lipolysis in adipose cells, reduction of edema, telomerase activation in skin cells, skin hydration improvement, skin roughness improvement, skin pigmentation improvement and skin tone improvement.

3. The kit of claim 1, wherein A) the telomerase activating composition increases the expression of one or more telosome subunit proteins selected from TRF1 (Telomeric Repeat Factor 1), TRF2 (Telomeric Repeat Factor 2), RAP1 (Repressor Activator Protein 1), TIN2 (TERF1-interacting nuclear factor 2), POT1 (Protection of telomeres 1), and TPP1 (Tripeptidyl peptidase 1) in skin cells.

4. The kit of claim 1, wherein A) the telomerase activating composition comprises a Dendropanax morbifera Lev. extract, a Kappaphycus alvarezii extract, or their mixture as the active ingredient.

5. The kit of claim 1, wherein application of A) the telomerase activating composition is combined with a treatment with an ultrasound laser having a frequency of 10 to 90 Hz.

6. The kit of claim 1, wherein B) the skin dermis irradiating laser is one or more selected from a laser having a frequency of 2 to 100 Hz and an RF (Radio Frequency) laser which deliver heat energy to dermis or to dermis and SMAS (Superficial Musculoaponeurotic System).

7. The kit of claim 6, wherein B) said skin dermis irradiating laser is one or more lasers selected from the group consisting of a laser having a frequency of 5 Hz, a laser having a frequency of 30 to 50 Hz and an RF laser having a frequency of 35 to 45 MHz.

8. The kit of claim 1, wherein C) the composition for topical fat removal comprising 300 IU to 600 IU of hyaluronidase is an injection composition.

9. The kit of claim 8, wherein the injection composition for topical fat removal further comprises one or more of a local anesthetic, an antihistamine, a lipolysis stimulator and a collagen production stimulator.

10. The kit of claim 9, wherein the local anesthetic is lidocaine, the antihistamine is Peniramin, the lipolysis stimulator is L-carnitine and the collagen production stimulator is vitamin C.

11. The kit of claim 10, wherein the injection composition for topical fat removal comprises 300 IU to 600 IU of hyaluronidase per single dose volume and, based on the weight of the total composition, 0.08 to 0.4% by weight of lidocaine as local anesthetic, 0.01 to 0.02% by weight of Peniramin as antihistamine, 0.01 to 4.0% by weight of L-carnitine as lipolysis stimulator, and 0.1 to 2.0% by weight of vitamin C as collagen production stimulator, with the remainder being saline solution, and the composition is injected at a single dose volume of 0.5 to 2 cc with an interval between injection points of 0.5 to 1.5 cm.

12. The kit of claim 11, wherein the injection composition for topical fat removal further comprises one or more components selected from phosphatidylcholine, aminophylline, caffeine, placenta and pentoxifylline in an amount of 0.01 to 4.0% by weight.

13. The kit of claim 1, wherein D) the skin muscle layer irradiating laser is irradiated from inside the mouth toward the face and is a laser having a frequency of 1 to 3.5 Hz which delivers heat energy to the skin muscle layer, or to the skin muscle layer and upper dermis.

14. The kit of claim 13, wherein D) the skin muscle layer irradiating laser is irradiated such that a temperature in the range of from 45 to 70□ is reached in the target skin layer.

15. An aesthetic system for the prevention or treatment of skin aging, comprising
A) a telomerase activating composition;
B) a skin dermis irradiating laser;
C) a composition for topical fat removal comprising 300 IU to 600 IU of hyaluronidase; and
D) a skin muscle layer irradiating laser.

16. The aesthetic system of claim 15, further comprising an ultrasound laser having a frequency of 10 to 90 Hz together with A) the telomerase activating composition.

17. The aesthetic system of claim 15, wherein B) the skin dermis irradiating laser is one or more selected from a laser having a frequency of 2 to 100 Hz and an RF (Radio Frequency) laser which delivers heat energy to dermis or to dermis and SMAS.

18. The aesthetic system of claim 15, wherein C) the composition for topical fat removal is an injection composition and further comprises a local anesthetic, an antihistamine, a lipolysis stimulator and a collagen production stimulator.

19. The aesthetic system of claim 18, wherein C) the injection composition for topical fat removal comprises 300 IU to 600 IU of hyaluronidase per single dose volume and, based on the weight of the total composition, 0.08 to 0.4% by weight of lidocaine as local anesthetic, 0.01 to 0.02% by weight of Peniramin as antihistamine, 0.01 to 4.0% by weight of L-carnitine as lipolysis stimulator, and 0.1 to 2.0% by weight of vitamin C as collagen production stimulator, with the remainder being saline solution, and the composition is injected at a single dose volume of 0.5 to 2 cc with an interval between injection points of 0.5 to 1.5 cm.

20. The aesthetic system of claim 15, wherein D) the skin muscle layer irradiating laser is irradiated from inside the mouth toward the face and is a laser having a frequency of 1 to 3.5 Hz which delivers heat energy to the skin muscle layer, or to the skin muscle layer and upper dermis.

21. An aesthetic process for the prevention of skin aging, wherein the following steps are carried out at least once irrespective of their order:
i) a step of applying a telomerase activating composition;
ii) a step of irradiating a skin dermis irradiating laser for 3 to 60 minutes;
iii) a step of injecting a composition for topical fat removal at a single dose volume of 0.5 to 2 cc with an interval between injection points of 0.5 to 1.5 cm; and
iv) a step of irradiating a skin muscle layer irradiating laser from inside the mouth toward the face for 3 to 10 minutes.

22. The aesthetic process of claim 21, further comprising irradiating an ultrasound laser having a frequency of 10 to 90 Hz for 5 to 20 minutes simultaneously with or immediately after step i).

23. The aesthetic process of claim 21, wherein the skin dermis irradiating laser in step ii) is one or more lasers selected from the group consisting of a laser having a frequency of 5 Hz, a laser having a frequency of 30 to 50 Hz and an RF laser, wherein
said laser having a frequency of 5 Hz is irradiated for 3 to 8 minutes,
said laser having a frequency of 30 to 50 Hz is irradiated for 15 to 40 minutes, and
said RF laser is irradiated at a frequency of 40.68 MHz for 4 to 10 minutes.
